(19) United States
(12) Patent Application Publication
(10) Pub. No.: US 2015/0248536 A1
(43) Pub. Date: Sep. 3, 2015

(54) MODULAR TELEMEDICINE ENABLED CLINIC

(71) Applicants: Jack TAWIL, Brooklyn, NY (US); Margaret TAWIL, New York, NY (US); Dov SANDBERG, Brooklyn, NY (US)

(72) Inventors: Jack Tawil, New York, NY (US); Margaret Tawil, New York, NY (US); Dov Sandberg, Brooklyn, NY (US)

(21) Appl. No.: 14/436,834

(22) PCT Filed: Oct. 21, 2013
(86) PCT No.: PCT/US13/65967

§ 371 Date: Apr. 17, 2015

Related U.S. Application Data
(60) Provisional application No. 61/716,383, filed on Oct. 19, 2012.

(51) Int. Cl. G06F 19/00

(52) U.S. Cl. CPC G06F 19/3418; G06F 19/345

(57) ABSTRACT

A modular medical clinic system that includes a patient check in module 10 for receiving patient data, the patient check in module 10 including video and audio input systems for delivering information to an information collection personnel 120 and a display module 30 for depicting a medical care provider 130 in real time to the patient within the modular medical clinic 100 and depicting the patient in real time to the remote medical care provider 130 during a medical diagnosis of the patient. The modular medical clinic system also includes an interface with inventory supply personnel and systems 140, and an interface with an operations personnel and systems 150.

### CROSS REFERENCE TO RELATED APPLICATIONS

This application claims the benefit of International Patent Application No. PCT/US2013/065967 filed on Oct. 21, 2013, titled “MODULAR TELEMEDICINE ENABLED CLINIC AND MEDICAL DIAGNOSTIC ASSISTANCE SYSTEMS”, which is incorporated herein by reference, and claims the benefit of U.S. Provisional Application No. 61/716,383 filed Oct. 19, 2012, titled “MODULAR TELEMEDICINE ENABLED CLINIC”, which is incorporated herein in its entirety by reference.

### BACKGROUND

1. Technical Field

The present disclosure relates to modular medical clinics, and more particularly to systems and methods of modular telemedicine enabled clinics.

2. Description of the Related Art

Traditional methods of dispensing healthcare are expensive. Scheduling appointments with physicians may be difficult, which may lead to extended periods of time from when a patient becomes sick to when the patient begins treatment. This in turn may exacerbate the illness, and increase the cost, both in price of treatment and in time spent with a doctor.

One response to the rising cost of healthcare has been staffing medical clinics with less expensive personnel. That is, replacing the doctors from which patients have traditionally received healthcare, nurse practitioners (NP), and physicians assistants (PA). Costs have also been reduced by decreasing the amount of time a patient spends with a healthcare provider.

However, using the foregoing techniques to reduce costs has been subject to increased resistance by practicing physicians, and organizations of physicians such as the American Medical Association (AMA). One reason for the resistance is due to a phenomena called lack of continuity of care. Lack of continuity of care may occur when a patient receives medical care from different healthcare providers that do not communicate with each other.

Traditional medical access points are time consuming and expensive to build. In lower populated areas, stick-built medical facilities may not be available or patients may have to travel great distances to reach suitable medical facilities. This can especially be the case for advanced and rare medical specialties. In these situations, the best practitioners can not provide their knowledge or care to the patient as being separated by geography.

### SUMMARY

In some embodiments, the methods, apparatus and systems that are disclosed herein may provide a network of modular medical clinics at physical locations that are equipped with location-appropriate interconnected medical diagnostic and lab tools supported by an internet-based provider network and systems for providing clinical decision support. The network may also function as a repository for medical images and data for patients. Further, the network extends to home based medical services and diagnostics with an interface that communicates with the network via the internet. In some embodiments, by applying artificial intelligence to such a database, a diagnostic tool set may be provided that can render diagnosis based on either collective doctor diagnosis or specific doctor diagnosis methods.

The physical modular medical clinics are to be built using a combination of purpose built modules, high definition teleconference equipment, flat panel, e.g., LED, displays and touch screen displays, in which the video conferencing aspects of the network may be in two or three dimensions. These structures are interconnected on board point of care and diagnostic equipment. The modular medical clinics will be powered by cloud based network allowing for the collection and exchange of video feeds, video call routing and a combination of real time and store-and-forward collection of diagnostic readings. This network may include video call queuing, and utilize a web native electronic medical records practice management solution that can allow for full interoperability. Thus, the engine will be able to communicate with disparate electronic medical record (EMR)/electronic health care record (EHR) systems to allow for true health data exchange and continuity of care.

In some embodiments, touch screens on the exterior of the modular medical clinic will have the ability to check in a patient, collect past medical history and provide a live remote video operator to assist the patient with scheduling, check in and payment (Insurance I Credit I Debit) similar to the front desk function in a standard clinic setting. The exterior may also include diagnostic equipment, such as blood pressure, otoscope and similar type devices, which may be used for screening purposes prior to or in lieu of admission of the patient into the modular medical clinic. In some embodiments, the exterior medical equipment will be in communication with a nurse or practitioner over the network. In some examples, the patient will have the ability to choose the next available doctor or wait for a specific provider/doctor of choice. The patient may have the option to select the doctor based on a variety of factors such as specialty, proximity to the clinic and so on. The patient may also be given the ability to choose their pharmacy of choice in the event they will need a script following the examination. In some embodiments, the video call and the patients file may then be held in a queuing system until it is the patient turn to see the physician/doctor.

The modular medical clinic may be prepped for each patient by a practitioner, such as a nurse Assistant (RN, LPN, MA or CNA). Once the modular medical clinic is ready, a screen mounted on the outside of the facility will display an identifier for the patient. This display can double as a digital sign carrying various information feeds including health tips, procedure pricing, health event info and related advertising. The system can also automatically send a text message or place an automated phone call to the patient's phone when they are next in line for use of the pod.

Once inside the pod, in one embodiment, the patient will be presented with on screen instructions to stand in a specified location, such as on a designated floor tile. In some embodiments, a combination of active sensors, which may include a millimeter wave scanner, and a floor scale can provide automated body mass index (BMI) information and capture accurate measurement information, and can provide a scanned body image of the patient. Such accurate measurements are possible with an active sensor technology, which does not require the removal of patient's clothes, and whose readings can assist in health and wellness programs. The Nurse Assistant may then ask the patient to be seated in an examination chair, at which time the nurse assistant may proceed to collect a variety of readings based on pre-defined protocols tied to the chief complaint. In some embodiments, all of the readings may automatically flow into the electronic medical record (EMR)/electronic health care records (EHR) system. In some examples, data collected from network may be transmitted through a user interface controlled by operations personnel or system that is located either at the modular medical clinic, the remote provider location or at a home location for the patient. In one embodiment, the Nurse Assistant may run through the initial protocols via a cantilevered tablet computer functioning as a control center.

This collected data will flow into the EMR and be presented to the provider/doctor once he/she accepts a patient. The doctor will have the chance to review the data prior to initiating the video part of the call. Once the video exam begins the doctor will either instruct the nurse to collect additional data or diagnostics or to leave the modular medical clinic so he may have a private conversation with the patient. If necessary the doctor will be able to notify the nurse that she is needed inside the pod by clicking on a button on his computer screen that will start a red light flashing outside the clinic. During consultation the provider/practitioner/health provider has access and control of the medical devices within the clinic in real time for diagnosing and/or treating the patient. Following the consultation, the doctor will be able to print a script and other appropriate medical data and condition appropriate coupons to the pods printer, or e-script direct to the pharmacy of choice. These prints can also be replaced by e-mail directly to the patient or storage of this data on the patient's online health portal.

In one embodiment, the methods and structures disclosed herein provide a modular medical clinic system for medical diagnostics that includes a patient check in module comprising a patient and information collection interface for receiving patient data from a patient being diagnosed in the modular medical clinic. The patient check in module includes video and audio input and output systems for delivering information to a remote information collection personnel in real time. The modular medical clinic system also includes a display module comprising a video display interface for depicting a medical care provider in real time to the patient within the modular medical clinic and depicting the patient in real time to a remote medical care provider during a medical diagnosis of the patient. In one embodiment, the modular medical clinic system also includes an inventory supply module with an inventory interface for receiving data on supplies used in the modular medical clinic. The inventory interface keeps track of the inventory used during examination and treatment of patients in the modular medical clinic system.

In another embodiment, a modular medical clinic system is provided that includes a plurality of modules. In some embodiments, the modular medical clinic system includes at least one sensor for taking at least one measurement from at least one module of the plurality of modules. In some embodiments, the modular medical clinic system further includes an interface with the at least one sensor for sending data from the at least one measurement to a remote operator.

In another embodiment, a diagnostic system is provided that includes a database comprised of comparative data collected from a plurality of patients. Each value of comparative data includes diagnosis for an ailment that is categorized by a diagnostic code. Each value of comparative data is also tagged with an institution type designation. The diagnostic system also comprises a diagnostic tool for taking a measurement of a patient. In some embodiments, the diagnostic system further comprises a comparison tool for extracting measurement data from the measurement of the patient and comparing the measurement data to the comparative data in the database by employing artificial intelligence. In some embodiments, a match determined by the artificial intelligence between the measurement data and the comparative data designates the measurement data with the diagnostic code as an initial diagnosis. The initial diagnosis may also include an expression of the number of matches for the comparative data to measurement data by the institution type designation. The diagnostic system also includes a display depicting the initial diagnosis including the expression of the number of matches for the comparative data to measurement data by the institution type designation. The institution type designation may include a designation of both institution and specific medical personnel.

In another aspect, a method of diagnosing a patient a patient is provided that includes providing a database comprised of comparative data collected from a plurality of patients, wherein each value of comparative data includes diagnosis for an ailment that is categorized by a diagnostic code, wherein each value of comparative data is tagged with an institution type designation. A measurement is taken from a patient, and measurement data is extracted from the measurement of the patient. The measurement data is compared to the comparative data in the database by employing artificial intelligence. A match determined by the artificial intelligence between the measurement data and the comparative data designates the measurement data with the diagnostic code as an initial diagnosis. The initial diagnosis also includes an expression of the number of matches for the comparative data to measurement data by said institution type designation.

In another aspect, a computer program product is provided for diagnosing a patient comprising a storage medium, the storage medium, not a signal, tangibly embodying a program of instructions executable by a computer, the program of instructions when executing performing the following steps that includes providing a database comprised of comparative data collected from a plurality of patients. Each value of the comparative data includes a diagnosis for an ailment that is categorized by a diagnostic code, and each value of comparative data is tagged with an institution type designation. Following steps include taking a measurement of a patient, and extracting measurement data from the measurement of the patient. A further step includes comparing the measurement data to the comparative data in the database by employing artificial intelligence, wherein a match determined by the artificial intelligence between the measurement data and the comparative data designates the measurement data with the diagnostic code as an initial diagnosis, wherein the initial diagnosis includes an expression of the number of matches for the comparative data to measurement data by said institution type designation. In a following step, the initial diagnosis is displayed including the expression of the number of matches for the comparative data to measurement data by the institution type designation.

In another embodiment, a diagnostic system is provided that includes a database of comparative data, wherein each data value in the database correlates a measured change in body dimensions and symptoms of the patient to a diagnostic code. Each value of comparative data is tagged with an institution type designation. In one embodiment, the system further includes a wave millimeter scanner for measuring changes in a patient's body geometry. A comparison tool is provided for comparing the changes in a patient body geometry and at least one patient symptom to the comparative data in the database by employing artificial intelligence, wherein a match determined by the artificial intelligence between the changes in a patient's body geometry and the at least one patient symptom and the comparative data designates the measurement data with the diagnostic code as an initial diagnosis. The initial diagnosis also includes an expression of the number of matches for the comparative data to measurement data by the institution type designation. The diagnostic system also includes a display depicting the initial diagnosis including the expression of the number of matches for the comparative data to measurement data by the institution type designation.

In another aspect, a method of diagnosing a patient is provided that includes providing a database comprised of comparative data wherein each data value in the database correlates a measured change in body dimensions and symptoms of the patient from which the change in body dimension was measured to a diagnostic code. The diagnostic code correlates to a disease or ailment. Each value of comparative data may also be tagged with an institution type designation. In some embodiments, the method further includes recording at least one symptom from a patient, and measuring changes in a patient's body geometry using a wave millimeter scanner. The method may further include comparing the changes in the patient's body geometry and at least one patient symptom to the comparative data in the database by employing artificial intelligence. A match determined by the artificial intelligence between the changes in a patient's body geometry and the at least one patient symptom and the comparative data designates the measurement data with the diagnostic code as an initial diagnosis. The initial diagnosis also includes an expression of the number of matches for the comparative data to measurement data by the institution type designation.

In another aspect, a computer program product is provided for diagnosis a patient comprising a storage medium, the storage medium, not a signal, tangibly embodying a program of instructions executable by a computer, the program of instructions when executing performing the following steps that include providing a database comprised of comparative data, wherein each data value in the database correlates a measured change in body dimensions and symptoms of the patient to a diagnostic code. Each value of comparative data is also tagged with an institution type designation. In a following step, at least one symptom from a patient is defined, and a change in a patient's body geometry is measured using a wave millimeter scanner. The steps further include comparing the changes in a patient's body geometry and at least one patient symptom to the comparative data in the database by employing artificial intelligence. A match determined by the artificial intelligence between the changes in a patient's body geometry and the at least one patient symptom and the comparative data designates the measurement data with the diagnostic code as an initial diagnosis. The initial diagnosis may also include an expression of the number of matches for the comparative data to measurement data by the institution type designation. In a following step, the initial diagnosis is displayed including the expression of the number of matches for the comparative data to measurement data by said institution type designation.

In another embodiment, a method of displaying medical records is provided that includes receiving medical records for a patient, and assigning data from the medical records to regions of a human body in which prior diagnosis and treatments were received by the patient. A body image of the patient may then be scanned. The body image of the patient may be displayed on a graphical user interface. An indicator is displayed on a portion of the body image correlating to the region of the human body that the data from the medical record has been assigned to. Selecting said indicator on the graphical interface displays the data on the diagnosis and treatments received by the patient for that region of the human body.

In another aspect, a computer program product for displaying medical records is provided that comprises a storage medium, the storage medium, not a signal, tangibly embodying a program of instructions executable by a computer, the program of instructions when executing performing the following steps that include receiving medical records for a patient, and assigning data from the medical records to regions of a human body in which prior diagnosis and treatments were received by the patient. In a following step, a body image of the patient is scanned. In a following step, the body image of the patient is displayed on a graphical user interface. The steps executed by the computer program product may further include displaying an indicator on a portion of the body image correlating to the region of the human body that the data from the medical record has been assigned to, wherein selecting the indicator on the graphical interface displays the data on the diagnosis and treatments received by the patient for that region of the human body.

In another aspect, a system for displaying medical records is provided that includes a database of medical records for a patient, wherein data from the medical records are assigned to at least one region of a human body in which prior diagnosis and treatments were received by the patient. The system may also include a wave millimeter scanner for scanning a body image of the patient. In some embodiments, a graphical interface is provided for displaying the body image of the patient and displaying an indicator on a portion of the body image correlating to at least one region of the human body that the data from the medical record have been assigned to in the database. In some embodiments, selecting the indicator on the graphical interface displays the data on the diagnosis and treatments received by the patient for that region of the human body.

In another embodiment, a medical diagnostic system is provided that includes at least one device taking at least one measurement from a patient, and an interface including a first connection to a display for providing video conferencing. The interface also includes a second connection for receiving data from the at least one sensor, and a third connection to a remote operator. Data from the at least one sensor is forwarded to the remote operator, wherein the remote operator communicates information from the at least one sensor to a remote provider, e.g., provider, and provides said video conferencing between the patient and the provider.

### BRIEF DESCRIPTION OF DRAWINGS

The disclosure will provide details in the following description of preferred embodiments with reference to the following figures wherein:

FIG. 1 is a top down schematic of a modular medical clinic, in accordance with one embodiment of the present disclosure.

FIG. 2A is a perspective front view of the exterior of the modular medical clinic depicted in FIG. 1.

FIG. 2B is a perspective side view of the exterior of the modular medical clinic depicted in FIG. 1.

FIG. 2C is a perspective top down view of the exterior of the modular medical clinic depicted in FIG. 1.

FIG. 2D is a perspective rear view of the exterior of the modular medical clinic depicted in FIG. 1.

FIG. 3A is a perspective view of a front interior wall including the door assembly of the modular medical clinic depicted in FIG. 1.

FIG. 3B is a perspective view of a side interior wall including a display module and a lab module of the modular medical clinic depicted in FIG. 1.

FIG. 3C is a perspective view of a rear interior wall including an inventory module of the modular medical clinic depicted in FIG. 1.

FIG. 3D is a perspective view of a side interior wall including a primary care module of the modular medical clinic depicted in FIG. 1.

FIG. 4A is a front view of the patient check in module with the door open to depict the patient interface, in accordance with one embodiment of the present disclosure.

FIG. 4B is a side cross sectional view of the structure depicted in FIG. 4A.

FIG. 5A is a perspective front view of a display module, as used in at least one embodiment of the modular medical clinic that is depicted in FIG. 1.

FIG. 5B is a side cross sectional view of the structure depicted in FIG. 5A.

FIG. 6A is a perspective front view of a dressing room module, as used in at least one embodiment of the modular medical clinic that is depicted in FIG. 1.

FIG. 6B is a side cross sectional view of the structure depicted in FIG. 6A.

FIG. 7A is a perspective front view of a laboratory module, as used in at least one embodiment of the modular medical clinic that is depicted in FIG. 1.

FIG. 7B is a side cross sectional view of the structure depicted in FIG. 7A.

FIG. 8A is a perspective front view of an inventory module, as used in at least one embodiment of the modular medical clinic that is depicted in FIG. 1.

FIG. 8B is a side cross sectional view of the structure depicted in FIG. 8A.

FIG. 9A is a perspective front view of a cabinet for housing a portable sink, as used in at least one embodiment of the modular medical clinic that is depicted in FIG. 1.

FIG. 9B is a side cross sectional view of the structure depicted in FIG. 9A.

FIG. 10A is a perspective front view of a primary care/nurse station module, as used in one embodiment of the modular medical clinic that is depicted in FIG. 1.

FIG. 10B is a side cross sectional view of the structure depicted in FIG. 10A.

FIG. 11A is a perspective front view of a storage module, as used in one embodiment of the modular medical clinic that is depicted in FIG. 1.

FIG. 11B is a side cross sectional view of the structure depicted in FIG. 11A.

FIG. 12A is a perspective front view of an examination chair module, as used in one embodiment of the modular medical clinic that is depicted in FIG. 1.

FIG. 12B is a side cross sectional view of the structure depicted in FIG. 12A.

FIG. 13 is a schematic view of one embodiment of a system for medical diagnostic, in accordance with one embodiment of the present disclosure.

FIG. 14 is a block/flow diagram illustrating one embodiment of a method for providing medical diagnostics and treatment with a modular medical clinic, in accordance with one embodiment with the present disclosure.

FIG. 15 is a screen shot of one embodiment of an introduction screen to the patient data entry portion of the method for providing medical diagnostics and treatment, in accordance with one embodiment with the present disclosure.

FIG. 16 is a screen shot of for registration of a new patient to the modular medical clinic, in accordance with one embodiment with the present disclosure.

FIG. 17 is a screen shot of a data entry page for entry of insurance information of the patient to be treated by the modular medical clinic.

FIG. 18 depicts one embodiment of a screen shot from the touch screen computer, on which the patient to be diagnosed/treated by the modular medical clinic may enter the chief complaint for which the visit to the modular medical clinic is being made.

FIG. 19 depicts the screen shot that displays the estimated charges for the treatment/diagnosis in response to the chief complaint to be charged by the patient for the visiting the modular medical clinic, as well as an estimated time for treatment/diagnosis of the patient.

FIG. 20 depicts the screen shot for entry of the patient's payment method, in accordance with one embodiment of the present disclosure.

FIG. 21 is a block/flow diagram illustrating one embodiment of a method for providing medical diagnostics, in accordance with one embodiment with the present disclosure.

FIG. 22 depicts one embodiment of a screen shot for the portal to the remote provider, in which the screen shot is of a scanned body image of a person being diagnosed/treated by the modular medical clinic, in which portions of the scanned body image has icons of a graphic interface denoting areas of prior medical treatments of the patient.

FIG. 23 depicts one embodiment of a screen shot of electronic medical records that correspond to the icons of the graphical interface that are positioned on the scanned body image depicted in FIG. 22.

FIG. 24 depicts one embodiment of a screen shot of a provider portal depicting video conferencing with the modular medical clinic, in accordance with one embodiment of the present disclosure.

FIG. 25 is a block/flow diagram illustrating one embodiment of a method for providing medical diagnostics and treatment with a modular medical clinic, in accordance with one embodiment with the present disclosure.

FIG. 26 depicts one embodiment of a screen shot of the results of an initial diagnosis of a patient, in accordance with one embodiment with the present disclosure.

### DETAILED DESCRIPTION OF PREFERRED EMBODIMENTS

Detailed embodiments of the claimed structures, systems and methods are disclosed herein; however, it is to be understood that the disclosed embodiments are merely illustrative of the claimed structures and methods that may be embodied in various forms. Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment. In addition, each of the examples given in connection with the various embodiments are intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the methods and structures of the present disclosure. For purposes of the description hereinafter, the terms “upper”, “lower”, “right”, “left”, “vertical”, “horizontal”, “top”, “bottom”, and derivatives thereof shall relate to the embodiments of the disclosure, as it is oriented in the drawing figures.

The flowcharts and block diagrams in the figures illustrate the architecture, functionality, and operation of possible implementations of systems, methods and computer program products according to various embodiments of the present invention. In this regard, each block in the flowcharts or block diagrams may represent a module, segment, or portion of code, which comprises one or more executable instructions for implementing the specified logical function(s). It should also be noted that, in some implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

Aspects of the present disclosure may be described herein with reference to flowchart illustrations and/or block diagrams of methods, systems and/or computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

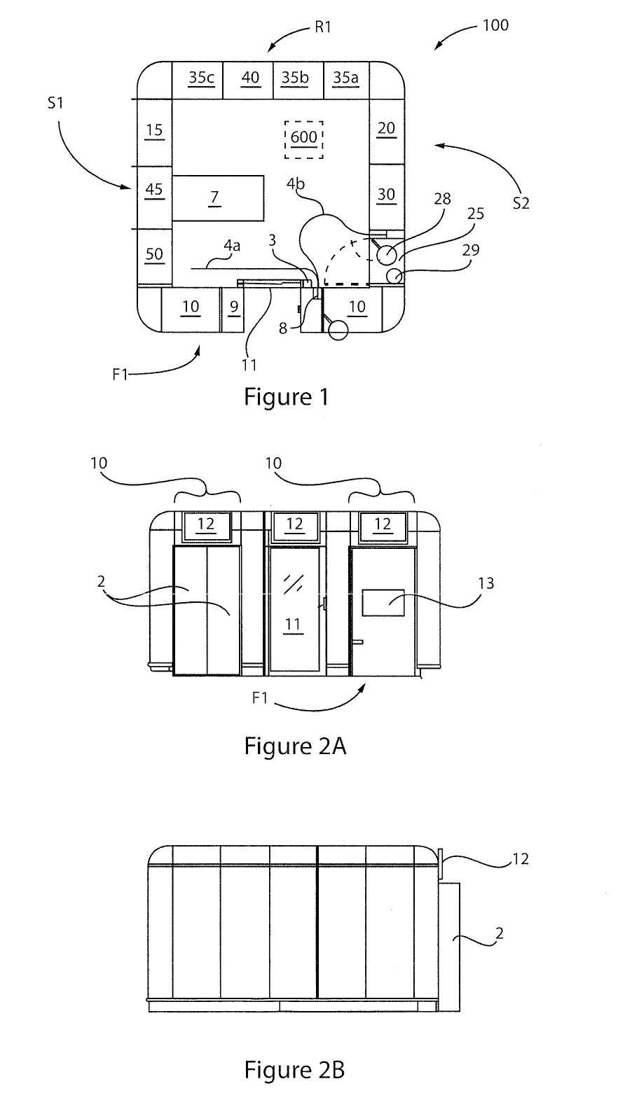

In one embodiment, the methods, systems, and apparatus that are disclosed herein provide a system that enables a delivery of high quality healthcare on a lower cost than previously possible by reducing fixed labor costs and employing a cloud based network to operate every aspect of a modular medical clinic that includes inventory supply, operations control, patient medical data entry and storage, billing, marketing, and other services associated with clinic management and delivery of medical services to patients. In some embodiments, the methods, systems and apparatus that are disclosed herein provide a modular telemedicine enabled clinic that may be powered by a network, such as a cloud based support system. Referring to FIG. 1, in one embodiment, the modular medical clinic 100 can be manufactured as a series of interconnecting modules. By “modular” it is meant that components of the modular medical clinic 100 are separately built structures, i.e., modules, including systems specific to the module that can be transported separately to a building location, and then interconnected to provide a housing structure. In some embodiments, the modules may be collapsible. For example, the collapsible feature may be enabled by a series of hinged attachments. For example, in one embodiment, the modular medical clinic 100 may include modules, such as, a patient check in module 10, a primary care module 15, a lab module 20, an inventory module 35a, 35b, 35c, a display module 30, and a dressing room module 25, wherein at least some of the modules are in communication with a telemedicine control system, as depicted in FIGS. 1-12B. In some embodiments, the telemedicine control system may be cloud based. The term “cloud based” is intended to describe a plurality of computers connected through a real-time communication network, such as the Internet. Cloud computing is a synonym for distributed computing over a network and means the ability to run a program on many connected computers at the same time. The phrase also refers to network-based services that appear to be provided by real server hardware, which in fact are served up by virtual hardware, simulated by software running on one or more real machines.

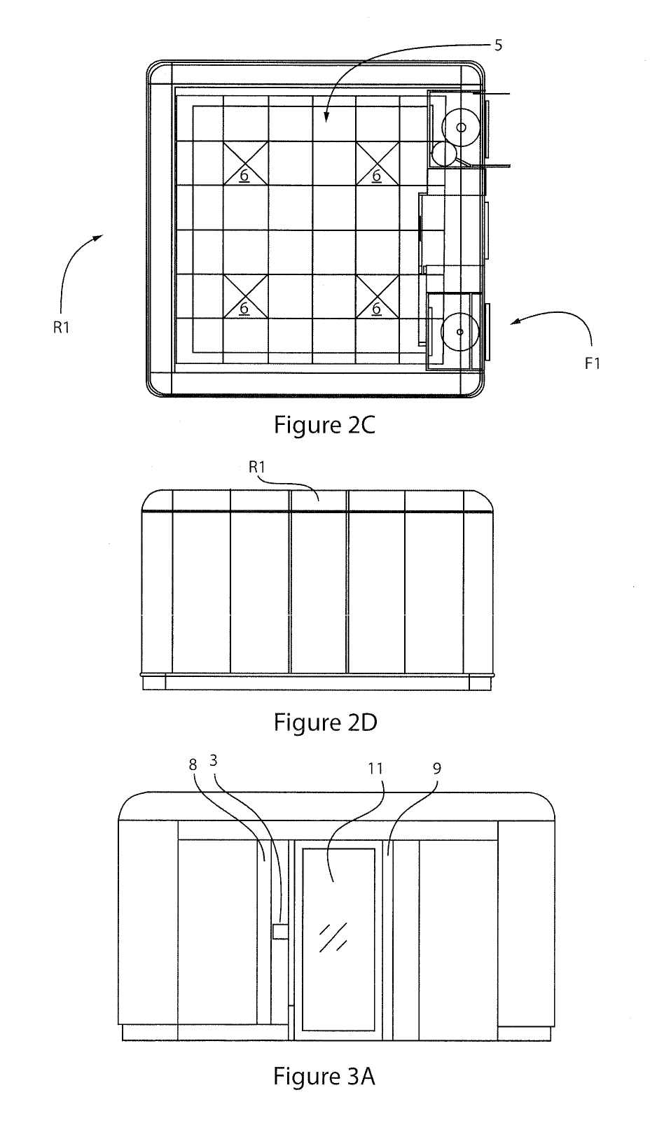

In some embodiments, each separate module may have dimensions on the order of 24″, 36″ and 48″. In some example, each panel may be at a standard 84″ high. The modules will have depths of 12″ and 24″. The above noted dimensions are provided for illustrative purposes only and are not intended to be limiting, as other dimensions are equally suitable for use with the structures disclosed herein. Lighting for the modular medical clinic 100 may be integrated into the top of at least one of the modules. A domed roofing system can rise up an additional 12″ to 24″ and consist of a sound proof material roofing system, which can allow for water to penetrate from a sprinkler system. FIG. 2C depicts a domed roofing system 5 that includes lighting elements 6, such as fluorescent lighting fixtures, integrated therein. Doors and access panels will be accessed via the interior of the modular medical clinic 100 with fixed back portions of the panels articulating towards the exterior. In some embodiments, each individual module is self-standing and is to be fixed to an adjacent module via a bracket system on the top and/or back side (fixed back side). In some examples, in order to give the appearance of a rounded or curved exterior, the system will employ metal framing similar to that of a round trampoline frame affixed to the top and bottom of each panel).

In some embodiments, the frame will be affixed to the top and bottom center of each panel and form a T shape (with the bottom of the T fitting into a receptacle affixed to the center, top and bottom of the panel). In another embodiment, the panels that provide the modules can be joined by using a bracket system that can be mounted to the top of the panels and contain two or more receptacles oriented to accommodate both the T shaped wall frame as well as the roof framing. In some examples, varying the angle of the receptacle changes the profile of the resulting walls and roof. Depending on the position of the panel, in the overall building structure the length of the T will be adjusted longer on the center panel and shorter towards the ends. The top of the T shall be curved to form an arc. The arc on each panel should fit together using a male/female design. The resulting structure will allow for a material with a channel at the top and bottom to be hung in a taut manner.

This above described bracket system shall be employed on at least 3 sides, i.e., rear side R1 and sidewall sides S1, S2, of the resulting modular medical clinic 100 (also referred to as a pod) and utilize a single roll of material substrate to effectively give the appearance of a smooth single piece design. FIG. 2B illustrates one example of the exterior of a sidewall side S1 of the modular medical clinic 100. FIG. 2D illustrates one example of the exterior of a rear side R1 of the modular medical clinic 100. End pieces will be employed on each side of termination to give a clean finish. These end pieces will intersect with the forth side of the pod and allow for a gradual tapering off.

Referring to FIGS. 1 and 2A, the fourth side, i.e., front side F1, of the modular medical clinic 100 includes the doorway entry panels 11 and can employ a low rise I-shaped frame system that may sit substantially flush against the adjacent modules and not employing any arc. The doorway entry panels 11 may employ sliding doors for entry and exit into and out of the modular medical clinic 100. The front side F1 typically contains doorway entry panels 11, display monitors 12 and touch screen computers 13, so selection of the substrate material for the front side F1 must take cut outs for these structures into consideration. The substrate can take the form of many different types of material. For example, the substrate used in construction for forming the modules of the front side F1 of the modular medical clinic 100 may be acoustic grade Polyurethane foam that can provide studio grade sound absorption or Neoprene, which is another insulating noise dampening material. In another embodiment, the substrate material may include a 3M wall decorating film on a sound substrate, which may allow for custom wrap applications. A system to allow for an easy change out of custom printed wraps or signage may also be incorporated into the design.

FIG. 2A depicts the exterior of the front side of the modular medical clinic 100, which includes modules for the doorway entry panels 11, and patient check in modules 10 on each side of the modules for the doorway entry panels 11. The patient check in module 10 that is present on the left side of the modules for the doorway entry panels 11 has the doors 2 closed, while the patient check in module 10 that is present on the right side of doorway entry panels 11 has the doors open to depict a handicap accessible touch screen computer 13, which is a component of the patient interface with a remote live medical information collection personnel.

Although FIG. 2A depicts two patient check in modules 10, wherein one patient check in module 10 is present on each side of the modules for the doorway entry panels 11, embodiments have been contemplated, in which only one patient check in module 10 is present in the modular medical clinic 100.

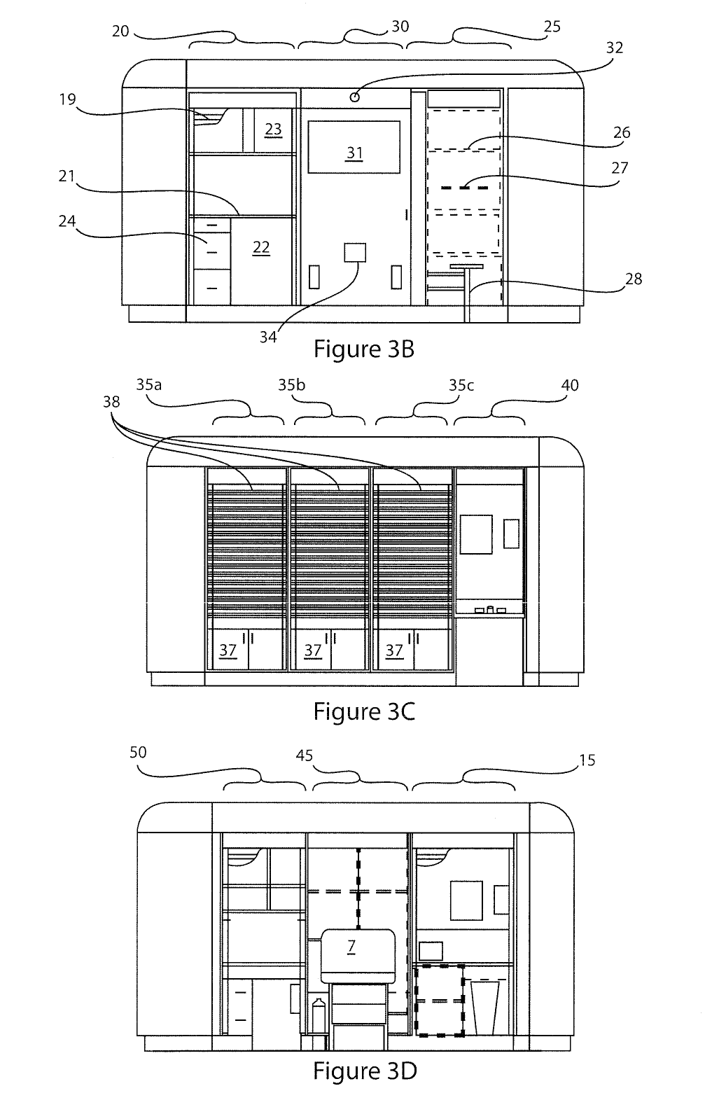

FIG. 3A is a perspective view of an interior wall of the modular medical clinic 100 including the doorway entry panels 11 from the perspective of the examining table 7 that is substantially centrally positioned within the modular medical clinic 100, as depicted in FIG. 1. In some embodiments, each door is approximately 18″ wide and the total door size is approximately 36″ when both doors are open. In some embodiments, the doors are controlled via push button, and may have a swooshing sound when opening to simulate the feeling of being air tight. In some embodiments, the door entry panels 11 include a red and green light on the outside, which can be used by the patient to indicate when they are finished changing, as well as being used by the doctor/physician/provider to indicate when the on-site nurse is to enter the interior of the modular medical clinic 100, and when the on-site nurse is not to enter the interior of the modular medical clinic 100. In some embodiments, the light is computer controlled so the remote provider can remotely turn it on and off.

Adjacent to the doorway entry panels 11 are storage closets 9 that may be used to house janitorial supplies for the modular medical clinic. On the opposite side of the doorway entry panels 11, is a curtain compartment 8 for housing a privacy curtain when not in use. Curtain tracks 4a, 4b are depicted in FIG. 1 defining the path that the curtain travels along to provide privacy regions. For example, a first track 4a positioned the curtain to extend past the doorway entry panels, which completely obstructs vision from the exterior of the modular medical clinic 100 into the interior of the modular medical clinic 100. A second track 4b positions the privacy curtain to provide a region adjacent to a dressing room module 25, in which the patient may change their clothes in privacy with access to the dressing room module 25. A control panel 3 is also present on the interior wall of the modular medical clinic 100 including the doorway entry panels 11. The control panel 3 may include lighting controls for the modular medical clinic 100.

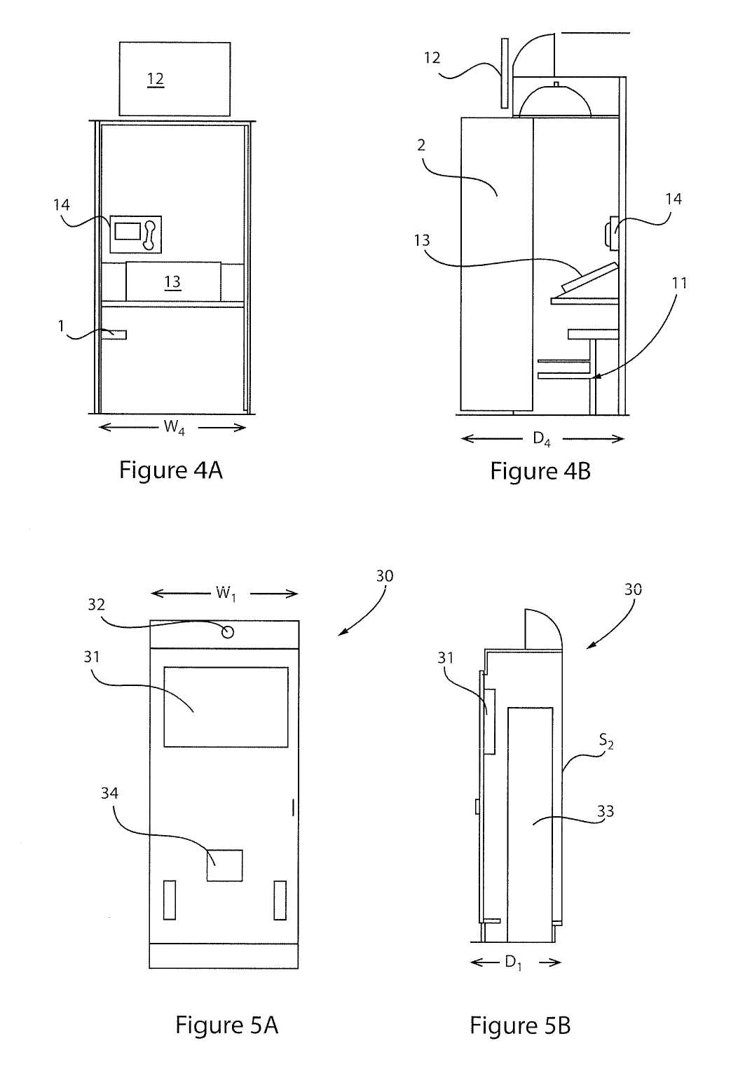

FIGS. 4A and 4B depict one embodiment of the patient check in module 10. In one example, the patient check in module 10 has an interior width W4 of about 42″, and an interior depth D4 of about 30″. In one embodiment, the patient check in module 10 may include a touch screen computer 13 and a telephone 14, such as a voice of internet protocol (VOIP) telephone. The patient check in module 10 also includes a privacy shield, i.e., cone of silence.

In some embodiments, the touch screen computer 13 is the interface that the patient checking into the modular medical clinic 100 for treatment and/or diagnostic enters their patient information, such as prior medical history, chief complaint, insurance information and method of payment. In some other embodiments, the computer for patient data entry may include a keyboard, which may be retractable, as an alternative to a touch screen interface.

A video phone is one embodiment of a type of telephone 14 that can provide the interface between the patient and a live remote medical information collection personnel. In some embodiments, it is intended that the live remote medical information collection personnel communicate through the video phone displaying an image of the remote medical information personnel to the patient with assistance and instructions to the patient for entering their medical information into the touch screen computer 13. In some other embodiments, the interface that the patient enters the patient data, e.g., medical history, into the network at the check in module 10 may include an interactive voice and video regions (IVVR) system. In some other embodiments, the phone may be eliminated and the live remove medical information personnel may communicate via video through the touch screen computer 13.

In some embodiments, the patient check in module 10 includes a seating apparatus, such as a stool 1, that is connected to at least one sidewall of the patient check in module 10. For example, the stool 1 may be pivotably connected to the patient check in module 10. The patient check in module 10 may also include voice recognition, video recognition, magnetic card swipe, scanner, biometric, facescan or similar identification technology.

A first sidewall of the modular medical clinic 100, e.g., the exterior sidewall S2, includes the display module 30, the laboratory module 20 and the dressing room module 25, as depicted in FIGS. 1, 2B and 3B. FIG. 3B in a perspective view of an interior wall of the modular medical clinic 100 including the display module 30, the laboratory module 20 and the dressing room module 25 from the perspective of the examining table 7 that is substantially centrally positioned within the modular medical clinic 100, as depicted in FIG. 1. From the perspective of the examining table 7, the display module 30 is positioned between the laboratory module 20 and the dressing room module 25.

FIGS. 5A and 5B depict one embodiment of a display module 30, as used in at least one embodiment of the modular medical clinic 100 that is depicted in FIG. 1. In one example, the display module 30 may have a width W1 ranging from 44″ to 48″, and a depth D1 ranging from 12″ to 24″. The display module 30 typically includes a display screen 31, such as a liquid crystal display (LCD) display screen, plasma screen, light emitting diode (LED) screen, or a combination thereof. In one example, the display screen 31 is provided by a 50″ LED screen measuring approximately 43.58″ wide×24.51″ high. The display screen 31 functions to display an image of a provider that is present at a remote location from the modular medical clinic 100 that is diagnosing and/or treating the patient within the modular medical clinic 100. The display module 30 also includes two dimensional and three dimensional capabilities. More specifically, in some embodiments, a real time three dimensional image of provider 130 may be depicted to the patient being treated in the modular medical clinic 10.

The display module 30 also includes at least one video camera 32. The video camera 32 may be a pan tilt video camera. The video camera 32 is typically present over the display screen 31. Alternatively, the video camera 32 is present under the display screen 31. In some embodiments, the video camera 32 is a high definition (HD) camera that provides 10× optical zoom, and support for Full 1080p HD up to 60 frames per second. Examples of video cameras 32 that are suitable for use with the display module 30 are available from Lifesize™. The video camera 32 functions to record the patient that is present in the modular medical clinic 100 to be viewed in real time by the provider that is present at a remote location from the modular medical clinic 100, in which the patient is being diagnosed and/or treated by the provider.

In some embodiments, the video camera 32 of the display module 30 has an automated cover (not shown) so as to give the patient the comfort that they are not being recorded while changing. In one example, the automated cover is housed in a compartment built into the interior wall of the display module 30 when the cover is in the open position for video conferencing. When the video camera 32 is not functioning in a filming or videoconferencing application, the cover extends from the housing to cover the video camera 32.

The display module 30 may also contain a computer system 33 that will power the equipment, such as the video conferencing equipment, that is present within the modular medical clinic 100. This can be housed inside the wall system with an access panel built into the wall near the floor. Typically, medical devices having USB interconnectivity that are used within the modular medical clinic 100 are to be connected with the computer system 33 that is housed within the display module 30. Therefore, the display module 30 may have multiple ports present therein having communication with the computer system 33 in order to accommodate the requirement that the medical devices can interface with the computer system 33. The display module 30 should also have internal and external power outlets. At least one panel of the display module 30 may have conduit space to run necessary wiring. The display module 30 can also accommodate all networking equipment both wireless and wired.

The display module 30 may also include a tablet computer 34 that may be cantilever mounted to the portion of the interior wall of the modular medical clinic 100 that is provided by the display module 30. The tablet computer 34 may be provided for entertaining the children of the patient being diagnosed and/or treated in the modular medical clinic 100. The tablet computer 34 may be mounted on a lower portion of the interior wall below the display screen 31.

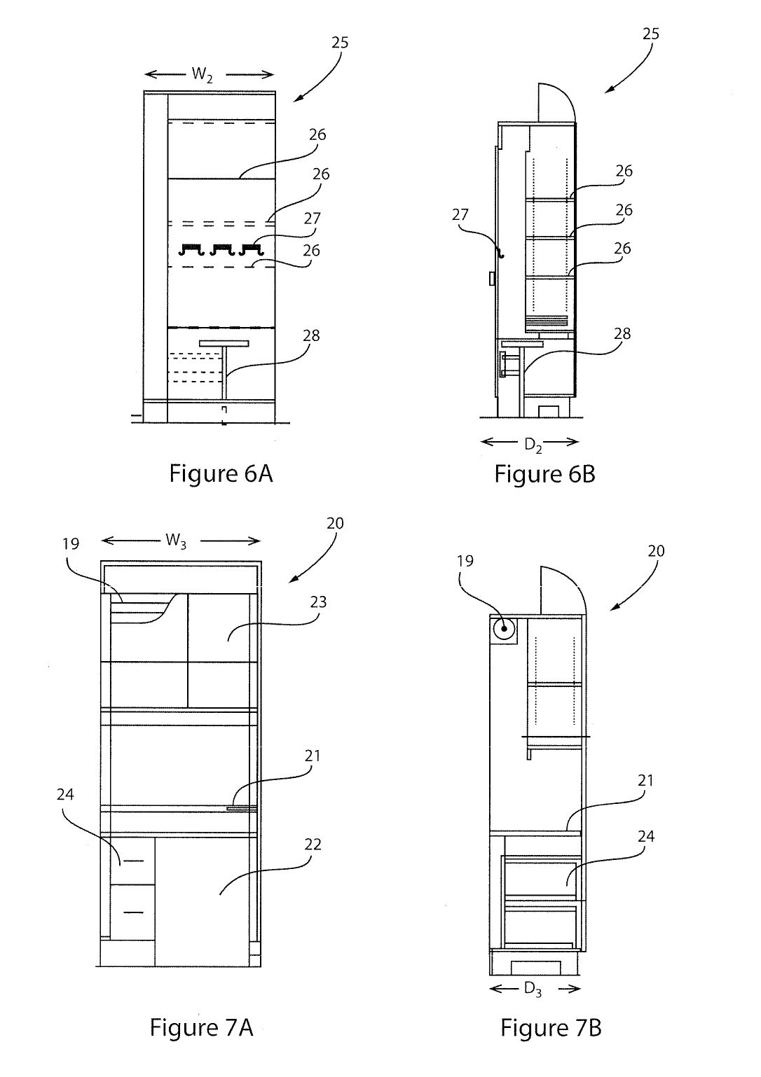

FIGS. 6A and 6B depict one embodiment of a dressing module 25 that is present adjacent to the display module 30 to provide a portion of one sidewall S2 of the modular medical clinic 100. In one embodiment, the dressing room module 25 is storage for a patient changing area, i.e., an area for a patient to get dressed and undressed for the purposes of examination. In one example, the dressing room module 25 has a width W2 of about 36″ and a depth D2 of about 12″. The dressing room module 25 may include a storage area in the form of a closet having a door for storing the personal items and clothing of the patient during the patient's examination. The storage area may include a series of draws (not depicted) and shelves 26. The door may be connected to the dressing room module 25 by hinged attachment, and may include at least one hook 27 for hanging clothing articles. The hook 27 may present on the surface of the door that will position the clothing articles hanging thereon within the closet area when the door is in the closed position. In some embodiments, at least one mirror (not shown) is present mounted on the door and/or interior closet surfaces of the dressing room module 25.

The dressing room module 25 may also include a seating apparatus, such as a stool 28, that is connected to at least one sidewall of the dressing room module 25. For example, the stool 28 may be pivotably connected to the dressing room module 25 so that the stool 28 is stored within the closet area when the door of the dressing room module 25 is closed, and swings out from the closet area when the door dressing room module 25 is opened. Although FIGS. 6A and 6B only depict a single stool 28, the dressing room module 28 is not limited to only this embodiment. For example, referring to FIG. 1, a second stool 29 may also be present with the dressing room module 25.

FIGS. 7A and 7B depict one embodiment of a laboratory module 20 that is present adjacent to the display module 30 to provide a portion of one sidewall S2 of the modular medical clinic 100. In one example, the laboratory module 20 has a width W3 that is equal to approximately 36″ and a depth D3 that is equal to approximately 24″. The laboratory module 20 typically includes an integrated counter space 21 with connected laboratory equipment. For example, the laboratory equipment that is connected, e.g., mechanically fastened, to the counter space 21 may include autoclaves, microscopes, centrifuge, shakers, incubators and a combination thereof. The laboratory module 20 may include equipment for testing including complete blood count (CBC, CBC with differential, blood count), urinalysis; routine urinalysis, UA), pap smear, occult blood test (Hemocult), cholesterol level and a combination thereof. In some embodiments, the laboratory module 20 may also include a refrigerator/freezer 22. In one example, the refrigerator/freezer 22 is a draw style refrigerator/freezer. The laboratory module 20 may also include storages structures, such as at least one overhead cabinet 23 and at least one file cabinet 24. In some embodiments, roll down security shutters 19 are integrated into the laboratory module 30 to secure the laboratory equipment and any information that is present within the storage structures housed within the laboratory module 30. The roll down security shutters 19 may be motorized.

Referring to FIGS. 1, 2D, 3C, 8A, 8B, 9A and 9B, the rear side R1 of the modular medical clinic 100 may include inventory modules 35a, 35b, 35c and a cabinet for housing a portable sink 40, such as a rolling sink. Although FIG. 3C depicts three inventory modules 35a, 35b, 35c and a single cabinet for housing the portable sink 40, the present disclosure is not limited to only this embodiment. Additionally, the order or arrangement of the inventory modules 35a, 35b, 35c and the cabinet for housing the portable sink 40 may be modified from the embodiment that is depicted in FIG. 3C. For example, the cabinet for housing the portable sink 40 may be present between the two inventory modules 35c, 35b, as depicted in FIG. 1. It has also been contemplated that a toilet (not shown) be integrated into the cabinet for housing the portable sink 40.

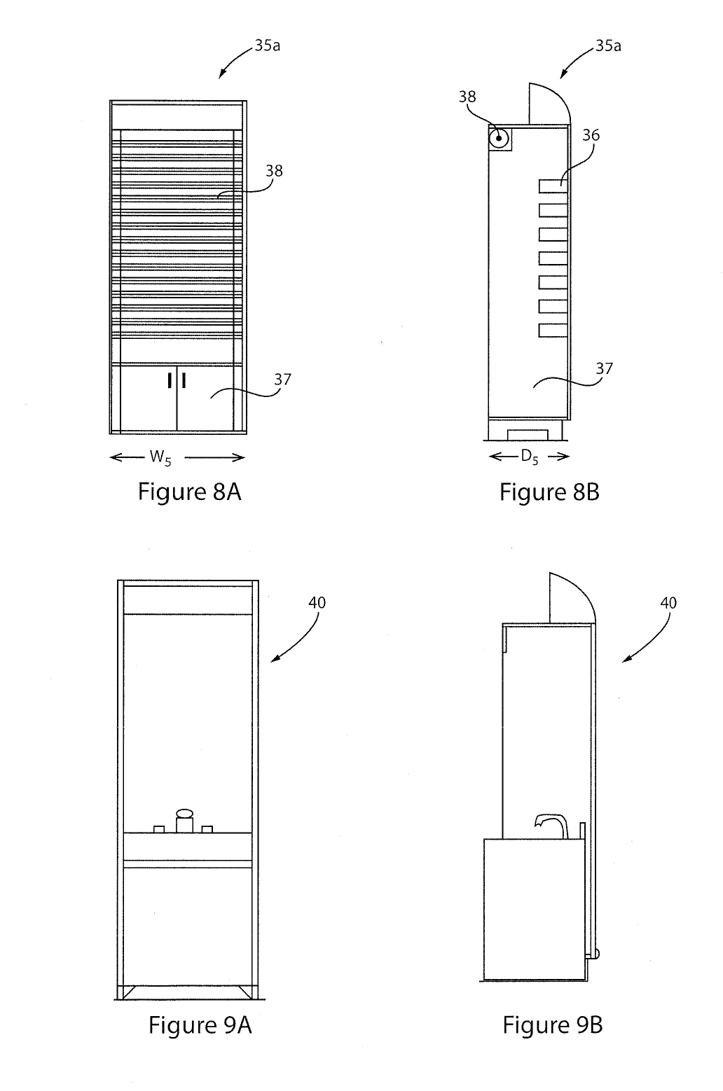

FIGS. 8A and 8B depict one embodiment of an inventory module 35a as used in at least one embodiment of the modular medical clinic that is depicted in FIG. 1. In one example, each of the inventory modules 35a has a width W5 on the order of 36″ and a depth D5 on the order of 24″. In some embodiments, the inventory modules 35a each include a shelving system that provides flexible storage ability. For example, the shelving system may include a plurality of bins 36, wherein each bin 36 may be labeled to designate its content. In some embodiments, a lighting system may be integrated with the shelving system to provide that specific bins 36 may be individually designated by being illuminated. In this manner, during a treatment or a diagnosis, the bins 36 containing the contents needed for the treatment are selected by the provider and illuminated. The illumination identifies the bins 36 containing the inventory that is needed by the nurse to diagnosis or treat the patient following the instruction of a remote provider. In some embodiments, a full cabinet, i.e., inventory module 35a, can hold between forty and fifty bins 36.

In some embodiments, a storage area in the form of cabinets 37 may be present in the inventory modules 35a, 35b, 35c below the shelving units containing the plurality of bins 36. In some embodiments, a step stool (not shown) may also be integrated into the bottom of the inventory module 35a to allow for easy access to bulk storage area at the top of the inventory module 35a. In some embodiments, roll down security shutters 38 are integrated into the laboratory module 30 to secure the inventory within the inventory module 35a, 35b, 35c. The roll down security shutters may be motorized.

FIGS. 9A and 9B depict a cabinet for housing a portable sink 40, as used in at least one embodiment of the modular medical clinic that is depicted in FIG. 1. In one example, the cabinet for housing a portable sink 40 has a width on the order of 36″ and a depth on the order of 24″. In some embodiments, the sink may be plumbed to the water supply of the building in which the modular medical clinic 100 is constructed, or the sink may have its own independent water supply.

A second sidewall of the modular medical clinic 100, e.g., the exterior sidewall S1, includes the nurse station/primary care module 15, the examination chair module 45 and storage module 50, as depicted in FIGS. 1, 2B and 3D. FIG. 3D in a perspective view of an interior wall of the modular medical clinic 100 including the nurse station/primary care module 15, the examination chair module 45 and storage module 50 from the perspective of the video camera 32 that is housed in the display module 30, as depicted in FIG. 1.

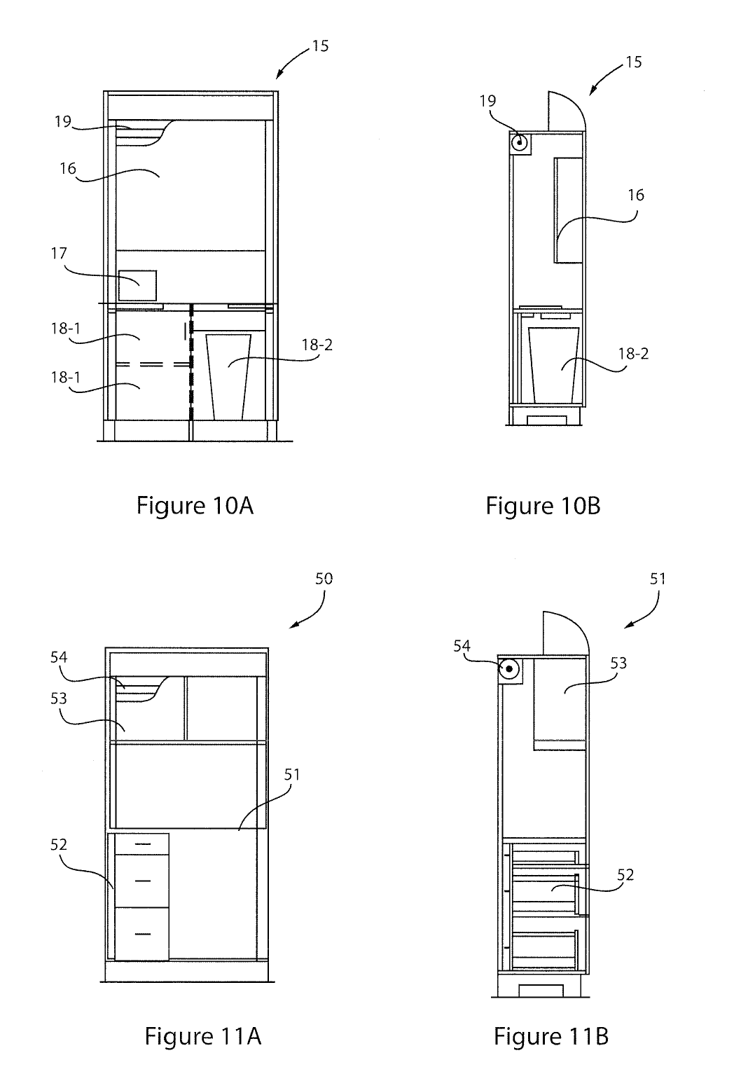

Referring to FIGS. 10A and 10B, in one embodiment, the nurse station/primary care module 15 includes a panel for mounting all of the primary care medical devices, which is hereafter referred to as a primary care panel 16. In one example, the width of the nurse station/primary care module 15 may be on the order of 36″ and the depth of the nurse station/primary care module 15 may be on the order of 24″. In some embodiments, the primary care panel 16 is electrified and includes electrical outlets. The medical devices may be tethered to the primary care panel 16, and the primary care panel 16 may extend in a telescoping arrangement from the primary care module 15 towards the area where the patient would be examined, e.g., on the examination table 7. For example, the primary care panel 16 may extend from the primary care module 15 by a dimension on the order of 3 feet or greater in a direction towards the examination table 7.

The medical devices that may be mounted to the primary care panel 16 include otoscopes, e.g., video otoscopes for examining the middle ear, exterior ear, nasal passages, mouth and throat in communication with the remote provider; stethoscopes, e.g., digital stethoscopes for evaluation of heart, lung and bowel sounds in communication with the remove provider, and exam cameras, etc. Other medical devices that may be mounted to the primary care panel 16 include thermometers (e.g., temperature taken via ear, temperature taken via mouth, thermometer to scan head or other area of body, etc.), oximeter (which measures the blood oxygen saturation), blood pressure cuff to measure blood pressure, electrocardiogram (EKG or ECG) (which provides a snapshot of the heart rhythm and data regarding stress or injury to the heart muscle), spirometer and transducer for measuring lung function, blood glucose measuring devices or monitors and retinal scan devices (e.g., Itronix retinal scan device, etc.). It is noted that the above list of medical equipment is illustrative and is not intended to be limiting. For example, the primary care panel may also include x-ray, ultrasound and bone density scanners. All of the equipment mounted to the will be integrated into the cloud based system so the remote provider will have access to images and data in real time.

In some embodiments, the nurse station/primary care module 15 also includes a nurse tablet computer 17 that may be mounted on a cantilever arm. The tablet computer 17 that is mounted to the nurse station/primary car module 15 is typically the nurse input device, in which the nurse may enter data that is transmitted to the provider as part of diagnosis or treating the patient and/or the supply personnel for providing information on the inventory of the modular medical clinic 100. The nurse station/primary care module 15 may also include a countertop positioned beneath the primary care panel 16, storage in the form of cabinets 18-1, such as file cabinets, and a pull out waste bin 18-2. The nurse station/primary care module 15 may also include cantilevered procedure tray (not depicted).

Similar to the inventory modules 35a, 35b, 35c, and the laboratory module 20, the nurse station/primary care module 15 may include roll down security shutters 19 that are integrated into the nurse station/primary care module 15 to secure the contents of the nurse station/primary care module 15 when not in use. The roll down security shutters 19 may be motorized.

Referring to FIGS. 11A and 11B, the storage module 50 may be similar in dimensions to the nurse station/primary care module 15. Similar to the nurse station/primary care module 15, the storage module 50 may also include a countertop 51, and storage cabinets, such as a file cabinet 52 and an overhead cabinet 53. The storage module 50 may also include roll down security shutters 54, which may be motorized.

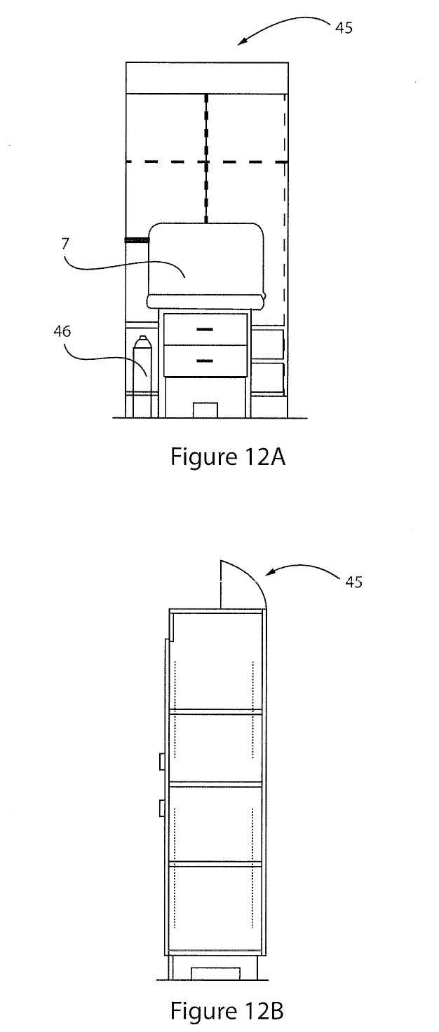

FIGS. 12A and 12B depict one embodiment of an examination chair module 45, as used in one embodiment of the modular medical clinic 100 that is depicted in FIG. 1. The examination chair module 45 typically includes storage, such as shelving and draws that may be positioned behind the examination table/chair 7. Although FIG. 12A depicts the examination table/chair 7 in front of the examination chair module 45, the examination table/chair 7 is typically a separate structure from the examination chair module 45. In some embodiments, gas containers, such as an oxygen tank 46 may be present within the examination chair module 45.

In some embodiments, a scale may be integrated into the floor of the modular medical clinic 100. The structure depicted in FIGS. 1-12B is entirely modular and designed to be assembled and disassembled within 24 hours. It is further noted that the material used in building the house for the each of the modules may be composed of injection molded plastics. It is also noted that the structure depicted in FIG. 1-12B is only one example of a modular medical clinic 100 in accordance with the present disclosure. For example, all of the dimensions are illustrative and not limiting. Further, other modules may be integrated into the modular medical clinic 100 that are not specifically depicted. Examples include X-ray enabling modules, cardiac modules, worker comp module, strength testing modules, ultrasound modules, imaging technology modules. Sub-specialties may also include, but it not limited to, pediatric, optimology, and other subspecialties.

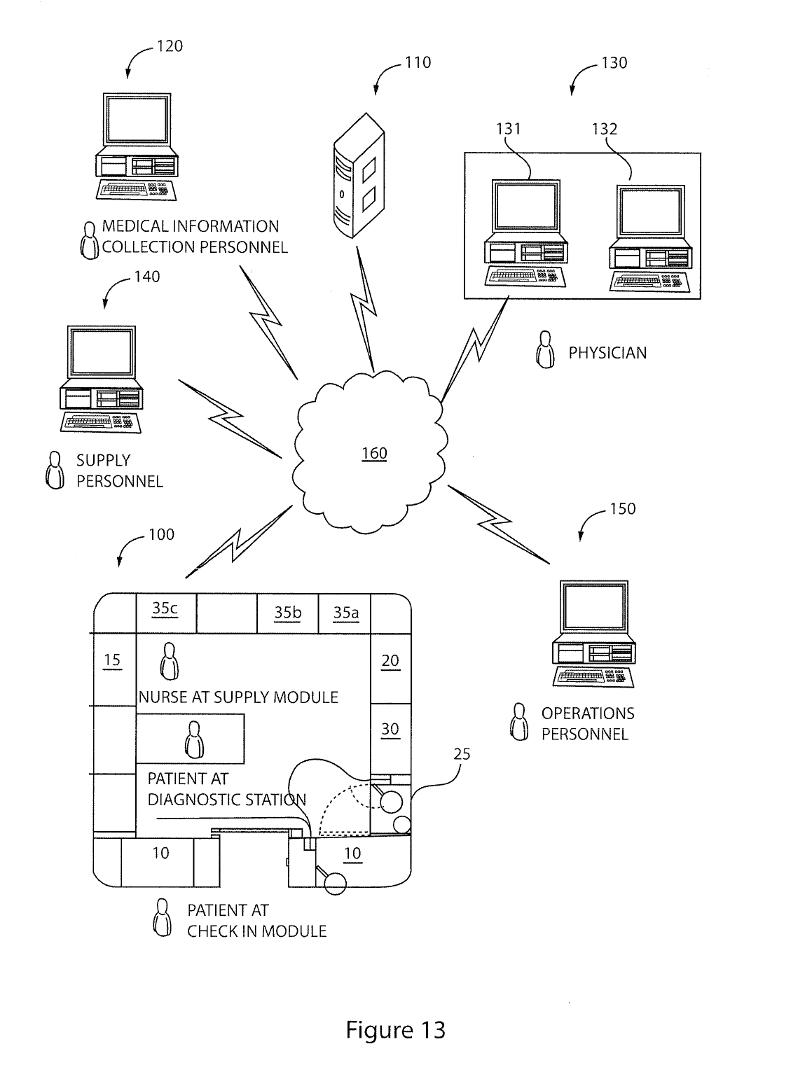

Referring to FIG. 13, the modular medical clinic 100 depicted in FIGS. 1-12B is integrated with a network, i.e., telemedicine control system. The network may include the modular medical clinic 100, at least one remote server 110, remote medical information collection systems and personnel 120, a remote provider and provider system 130, remote supply systems and personnel 140, remote operations personnel and systems 150 and a network connection system 160. The network may include a plurality of modular medical clinics 100. Each of the modular medical clinics 100 may be given an identification number which will enable it to interact with the network. In addition to the identification number each of the modular medical clinics 100 will have an assigned physical address.

The network connection system 160 may take a variety of forms including the Internet. When the network connection system 160 is, for example, the Internet, interaction with the modular medical clinic can be conducted from remote systems with remote personnel who are geographically situated at long distances from one another. Other forms of network connection systems that may also interact with the modular medical clinic 100 include local area network and wide area network systems.

The network may also include at least one remote server 130 that provides remote cloud capability, and can communicate with the network through the network connection system 160. For example, the at least one remote server 130 may include a CPU and memory.

The network may also provide for interconnectivity and communications between the modular medical clinic 100 and remote operations personnel and systems 150 for monitoring and maintaining optimal operation of the modular medical clinic 100. By maintaining the optimal operation, it is meant that the remote operations personnel and systems 150 can monitor operations systems within the modular medical clinic, such as lighting, display system performance, medical device performance, video camera performance, functionality of the medical devices on the primary care panel 16 of the primary care module 15, and also may control the security of the modular medical clinic 100. In some embodiments, the modular medical clinic 100 may include at least one sensor for taking at least one measurement of the performance of the modular medical clinic from at least one module of the plurality of modules, wherein the measurements taken by the sensor are transmitted over the network connection system 160 so that the performance of the modular medical clinic 100 may be monitored remotely by the remote operations personnel and systems 150.

In some embodiments, the operations personnel and systems 150 monitor the health of the communications cloud infrastructure to connect the patients of the modular medical clinic to the provider 130 via video, e.g., through the display module 30 of the modular medical clinic 100. The operations personnel and systems 150 also integrate with and monitor the performance of the display of data and video transmitted from the medical devices integrated with the primary care panel 16, as well as medical devices integrated with the laboratory module 20. The operations personnel and systems 150 may also administer the video call center functionality between the video phone 14 (or touch screen computer 13 when phone is not present) that is present in the check in module 10 and the medical information and collection personnel 120. In some embodiments, the operations personnel and systems 150 may also administer and monitor the performance of the communications system for the internal and external phone calls to the modular medical clinic 100.

The operations personnel and systems 150 may maintain the security of the modular medical system 100 through a security system that includes auto lock doors and internal and external security camera. For example, for the exterior of the facility, a camera should be mounted from the roof system showing top down view of the facility and its surrounding area. Interior security can be employed by re-purposing the existing camera 32 of the display module 30 for security check when not in use. Other systems of the modular medical clinic 100 controlled by the operations personnel and systems include airflow control, e.g., HVAC, electric and data control panel, and the advertising panel control for the exterior display displays 12.

In some embodiments, operations personnel and systems 150 can administer a health status check of the modular medical clinic 100 on a predetermined basis, e.g., at a time between 9 AM and 6 PM daily or every 3 hours etc. The system can verify all equipment is performing correctly, and issue an all systems OK status report when appropriate. If any issue is discovered, the system alerts the owner of the modular medical clinic 100 and operations personnel and systems 150.

The operations personnel and systems 150 provide the interface through which all data either taken from the patient at the modular medical clinic 100 and the web based home visit application is distributed to the other components of the network, such as the remote provider 130, the supply personnel and systems 140, medical information collection personnel and systems, as well as controlling the revenue cycle of the network. In some embodiments, the interface provided by the operations personal and systems 150 may include at least three connections. One connection is to the medical devices within the modular medical clinic. The other connection is to the video conferencing equipment. A third connection connects the interface provided by the operations personal and system 150 to the network connection system 160.

The medical information and collection personal 120 collect medical data including prior medical history from the patients that are to be diagnosed and/or treated in the modular medical clinic 100. For example, a patient may be able to do a complete check in view a live remote medical secretary located at a remote call center, identified as medical information and collection personal having reference number 120 in FIG. 13. The modular medical clinic 100 includes an interface for client check in, entry of patient identity, insurance information, medical history and chief complaint, in communication with the live remote medical secretary in the patient check in module 10, as depicted in FIGS. 1, 2A, 4A and 4B.

The remote provider 130 may have a work station, also referred to as a pod, that includes a personal computer with a form of memory, speakers and a high definition video camera. As used herein, the term “provider” denotes a physician, practitioner or doctor. In some embodiments, the work station of the remote provider 130 may be a dual screen set up, i.e., have a first station 131 and a second station 132, so that video information can be separated from screens including a high density of text. The remote provider 130 may conduct the examination by viewing the patient over the network in real time using the video systems 32 contained within the display module 30 of the modular medical clinic 100, as depicted in FIGS. 1, 3B, 5A and 5B.

Several of the medical tools connected to the primary care panel 16, such as exam camera (50× magnification) with integrated light source and tongue depressor, video otoscopes, digital stethoscope, digital x-ray, ultrasound and bone density scanners, ENT scope with built in light source, and dermascope, in the primary care module 15 are integrated into the cloud based system, i.e., network, so the remote provider 130 will have access to images and data collected using the instruments mounted to the primary care module 15 in real time. The remote provider 130 may also have access to the electronic medical record (EMR) for the patient that are provided by the patient either through the check in interface of the check in module 10 of the modular medical clinic 100, or given to the remote medical information check in personnel 130 by the patient. The remote provider 130 may also have access to prior electronic medical records of the patient, i.e., prior medical history, that are already stored with the network databases, e.g., stored on the at least one remote server 110. The remote provider 130 may also has have access to practice management and point of service (POS) systems that are integrated with the modular medical clinic, as well as an on site mini lab consisting of an array of integrated Clinical Laboratory Improvement Amendments (CLIA) waved and non-waved point of care devices. The remote provider 130 may also have access to any sensor based information that is collected.

The network may also include supply personnel and supply systems 140 for the modular medical clinic 100. The modular medical clinic 100 includes inventory supply modules 35a, 35b, 35c for containing medical supplies, i.e., inventory, and an inventory interface for receiving data on supplies used in the modular medical clinic 100. The portal to the inventory interface is to the provider 130, wherein the procedures and diagnosis selected through a computer interface by the provider 130 to be administered to the patient within the modular medical clinic 100 has a diagnostic code corresponding to a pick list. Selection of the diagnostic code by the provider 130 indicates a change in inventory of the items listed in the pick list that is present within the modular medical clinic 100. That change in inventory information is forwarded to the supply personnel and systems 140 through the operations personnel and systems 150. In some embodiments, the modular medical clinic 100 may employ an inventory control. In some embodiments, this inventory control will auto populate an inventory level. Once the inventory level falls below the minimum level it triggers an order to the supply personnel and supply systems 140. Communication between the personnel and supply systems 140 and the modular medical clinic 100 may be provided by a network connection system 160. All orders are batched to enable efficient shipment of orders. In some embodiments, inventory may be constantly monitored by the supply personnel and supply systems 140 through the interface with the operations personnel and operations systems 150, as such a critical inventory level can also be set. If such level is reached an immediate order for such stock keeping unit (SKU) will be issued and override the batching of such SKU. Further that SKU must be removed from the batched order to ensure no duplication of orders take place. In some embodiments, a module enables the modular medical clinic 100 owner to monitor inventory, prompts them to authorize orders prior to release and sets preauthorization parameters must be employed. The system can also be configured to automatically send resupply orders, with an override system by the owner of the modular medical clinic 100.

In some embodiments, the modular medical clinic 100 may deal with inventory using 2 storage methods—bin inventory and bulk open stock inventory. A predetermined number of bins will be found inside a specified “Bin Inventory” module or panel, i.e., inventory supply modules 35a, 35b, 35c. For example, the number of bins may number from 1-126 (based on current stock in list). All carried stock items will be assigned a bin number. Each bin # will be assigned a minimum and maximum part level. When the inventory level falls below the minimum level, a reorder will be placed. This reorder will be batched and await a predefined minimum or amount or specified order date. Bulk inventory will also be assigned a specific location within the “Bulk Inventory” module in line with the assigned bin number. Nurses will be charged with maintaining the proper balance between “Bin Inventory” and “Bulk Inventory” levels.

In some embodiments, the network should essentially be a portal that allows for the care givers, i.e., providers 130, to register on the system, i.e., register as being a care giver providing service through the modular medical clinic 100. For example, the providers 130 may designate their specialty, certifications, location, bill rates, type of payment accepted and types of insurance accepted. This information may be stored on the remote servers 110 of the network, and may be made available to patients intending to be diagnosed and/or treated in the modular medical clinic 100. In some embodiments, the operations personal 150 may verify whether that all licenses of the providers 130 participating in the network are in good standing. The network, e.g., the operations personnel and systems 150, can also collect the necessary information from the doctors to allow for credentialing to take place as well as accept payment from the providers 130 so that the providers 130 can get registered as participating parties with the modular medical clinic and credentialed on the system.

Typically, the network recognizes the location of the modular medical system 100 so as to comply with reimbursement scenarios specific to the location. Methods of payment must be location dependent, based on current and evolving reimbursement rules, e.g., medicare, medicaid, private insurance, cash, charge, debit, check and a combination thereof.

In some embodiments, the providers 130 can select a number of preferences on the portal, i.e., network, with the modular medical clinic 100 which should govern the way they are able to see patients in the modular medical clinic 100 and receive revenue on the system. In some embodiments, the providers 130 should be able to fill out a profile. In other embodiments, a providers' profile on the network may auto populate based on available info from the AMA, Heatthgrades or other similar databases, which may be internet accessible. The provider profile may allow potential patients of the modular medical clinic 100 to review the providers 130 prior to choosing them. Further, the network may assign a rating system and patient review feature to allow the patients pf the modular medical clinic 100 to rate their experience right at the end of the diagnosis and/or treatment in the modular medical clinic 100 or online after the patient has left the location that the modular medical clinic 100 is present at. At a glance the patient of the modular medical clinic 100 should be able to view of provider's 130 credentials both on line and off line and patient rating info.

Once the provider 130 is registered on the network they should be entered into a network contact list. This system will allow for patient interactions and tracking of such interactions. In addition to seeing patients in the modular medical clinic 100, the network may allow the providers 130 to connect to new and returning patients online for consultation and follow up's. As such, the network may include an online patient portal that can allow a patient to register for a consultation with the providers 130, enter payment information and connect to a provider. In some embodiments, if the patient is a returning patient the provider 130 will be able to see past medical history info from the patient's previous visit(s) to the modular medical clinic 100.

The providers 130 should be able to see all of the electronic medical record (EMR) information from the portal to the modular medical clinic 100, i.e., through the network communication system 50, as well as see the patient via video call, write an e-script and control other features remotely, such as camera zoom, pan, tilt and trigger the nurses call button. In some embodiments, the interface, e.g., provider's pod, used by the remote providers to interact with the modular medical pod 100 may include a first station 131 and a second station 132.

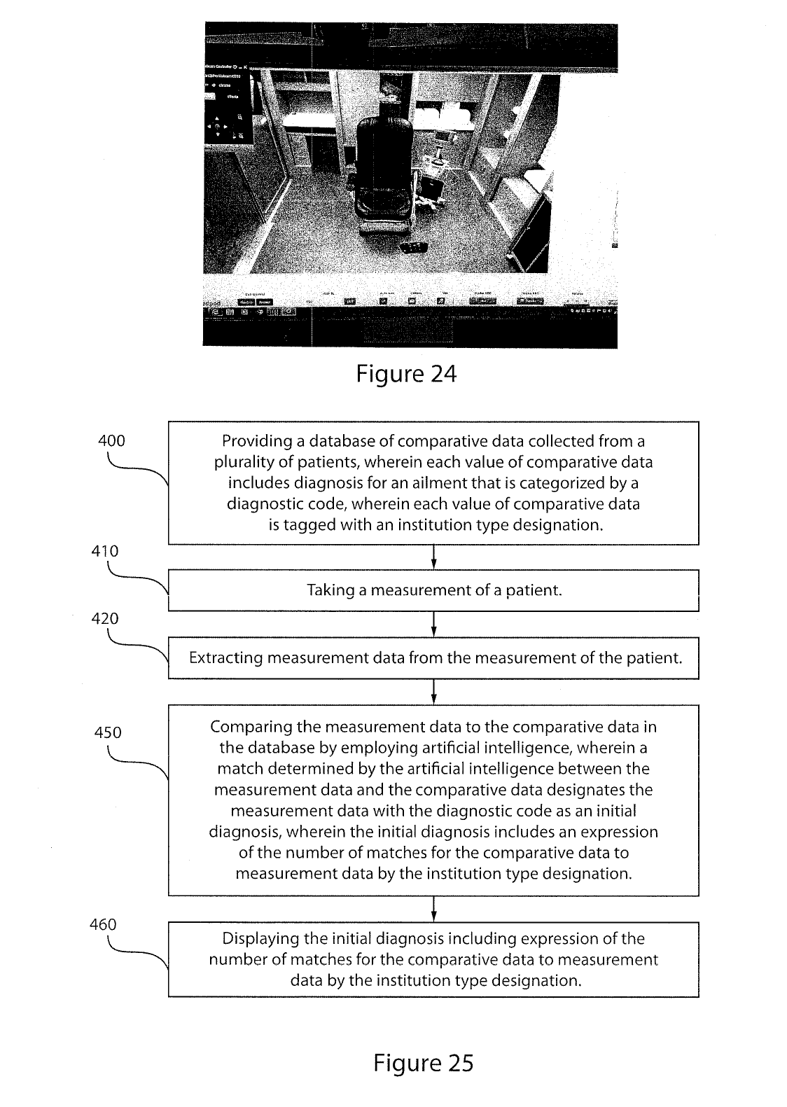

The first station 131 may display information received from the cameras, such as the video camera 32 in the display module 30, and information received from the medical devices that are connected with the primary care panel 16 of the primary care module 15. All information received from the primary care module 15 and sent to the remote provider 130 is sent through the operations personnel and systems 150 maintaining the interconnectivity of those systems. In some embodiments, the first station 131 may also display patient information that is entered into the network by the nurse into the nurse computer tablet 17. One example of a screen shot from the first station 131 of the providers interface, i.e., provider pod, depicting the examination table within the modular medical clinic 100 via the video call system, i.e., video conferencing, is depicted in FIG. 24.

The second station 132 of the interface, e.g., provider's pod, used by the remote providers to interact with the modular medical pod 100 may display patient information that has been provided to the medical information connection personnel and systems 120 through the check in module 10.

In some embodiments of the check in module 10, the interface that the patient enters the patient data, e.g., medical history, chief complaint, insurance information and method of payment, into may include at least one computer, such as a touch screen computer 13, and a communications device, such as a telephone 14 providing communication with the medical information connection personnel 120. In some embodiments, both video telephone functionality may be merged into the touch screen computer 13, so that the video telephone may be eliminated.

In some embodiments, the network may also provide for a web based portal, through which the patient and the provider may both have access to the network. A patient may access the network via a web site, i.e., web portal. The patient may access their medical chart from their visits to the modular medical clinic 100 through the web portal. The web portal may also allow credit card processing, and may allow a patient to view a provider roster display with rating system. The web site may also be used by the patient to enter past medical history into the network for inclusion into electronic medical records system. Provider information that may be viewed by a patient or prospective patient via the website may include states/jurisdictions/regions that a provider is licensed in, a provider specialty, educational background, patient generated ratings and comments. Patients may be able to log onto web site and sign up for an appointment to be seen by a provider 130. The patient should be able to see a provider on line via a “Virtual Medical Home” application, or in a modular medical clinic 100.

If the visit is scheduled to take place online, the website needs to enable a web cam. Data can also be collected by enabling a remote live agent to start the call with the patient, and physically enter certain data point prior to connecting to a provider 130 to the patient. In some embodiments, the web site allows the patient to directly share their medical records with a provider 130, includes a delivery mechanism such as e-mail or fax. The web site may also include a fee collection system to enable charges would be necessary based on delivery mechanism chosen.

In some embodiments, the web based portal for home visits may employ a medical diagnostic system including at least one device for taking at least one measurement from a patient, and an interface including a first connection to a display for providing video conferencing, a second connection for receiving data from the at least one sensor, and a third connection to remote operations personnel and systems. This medical diagnostic system is suitable for home applications, but may also be used in any location, and can provide for a portable means for a patient to received medical care.

In some embodiments, the at least one measurement may be taken using at least one of an otoscope, a wave millimeter scanner, a backscatter machine, an x-ray, an otoscope, a stethoscope, an exam camera, a video, a thermometers, an oximeter, a blood pressure cuff, an electrocardiogram, a spirometer, a transducer, a blood glucose measuring devices, scan devices and a combination thereof. Although some of the aforementioned devices are too large or complex for in home use, the operations personnel and systems 150.

In some embodiments, data from the at least one sensor is forwarded to the remote operator, e.g., remote operations personnel and systems 150. The remote operations personnel and systems 150 communicate information from that at least one sensor to a remote provider 130. The remote operator and systems 150 provides video conferencing between the patient and the remote provider 130 for medical diagnosis. In this embodiment, the display for providing the video conferencing may be a game console, a mobile device, a cellular device, or a personal computer having a web cam.

The web site may also smart phone application, that manages availability so a doctor can easily update availability on the system and receive notices in the notification bar of their smartphone indicating that a patient visit has been requested. This notice should go out to several available doctors and be issued to the doctor on a first come first serve basis.

The network may also include a medical resource locator application for smart phones, which will enable the patients to see the availability available doctors as well as enabling a secure 2 or 3 way communication between parties. This will enable doctors to install an application on their smart phone or various other access devices and register their availability on a general basis as well as check in as available and accept and deny requested appointments.

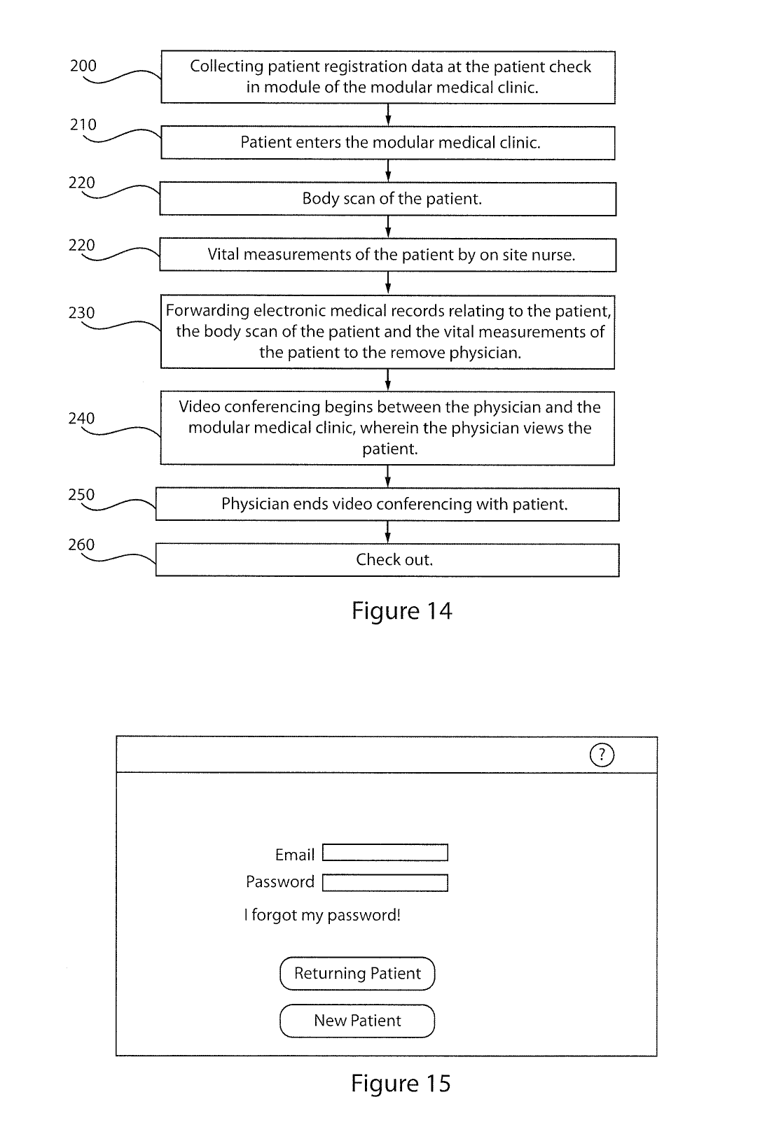

FIG. 14 illustrates one embodiment of a method of providing medical services using the modular medical clinic 100 depicted in FIGS. 1-13. The method may begin with collecting patient registration data at the patient check in module 10, e.g., at a patient check in Kiosk, of the modular medical clinic 100 at step 200. More specifically, as the patient enters the patient check in module 10, the patient may touch the screen of the touch screen computer 13, which can initiate the patient data entry process.

The entry of the patient data at the patient kioske portal is the first interaction of the patient with a web native, i.e., web based, electronic medical records system that includes emergency medical records (EMR)/electronic health care record (EHR)/medical billing (PM)/point of sale (POS) system that is integrated with the network including the modular medical clinic 100.

This includes patient kiosk check in as well as online check in capabilities, and can handle medical office processes including, scheduling, patient check in, insurance benefit verification, payment and co-payment processing etc. This system must also handle the medical protocols, patient health records, e-prescribing, lab interface and practice management functions. All of the medical diagnostic equipment, e.g., equipment connected to the primary care panel 16, and lab equipment that is integrated with the laboratory module 20 will communicate with this electronic medical records system. Data for the electronic medical records system may be stored on the at least one server 110 of the network, and may function.as a de facto hub. Such equipment interfaces must be completed based upon the list of equipment contained in the modular medical clinic 10, possibly using one aggregated data stream from all medical devices (MMDA—Mobile Medical Digital Assistant).

In some embodiments, the electronic medical record system must be interoperable, therefore allowing for the sharing of the patient encounter with the patient's doctors regardless of the medical records and/or medical history for the patients they may be using in their own practice. The electronic medical record system must allow for dual site log in enabling the on site nurses, in the modular medical clinic side 100 and the remote providers 130 to make annotations to the patients' file at the same time.

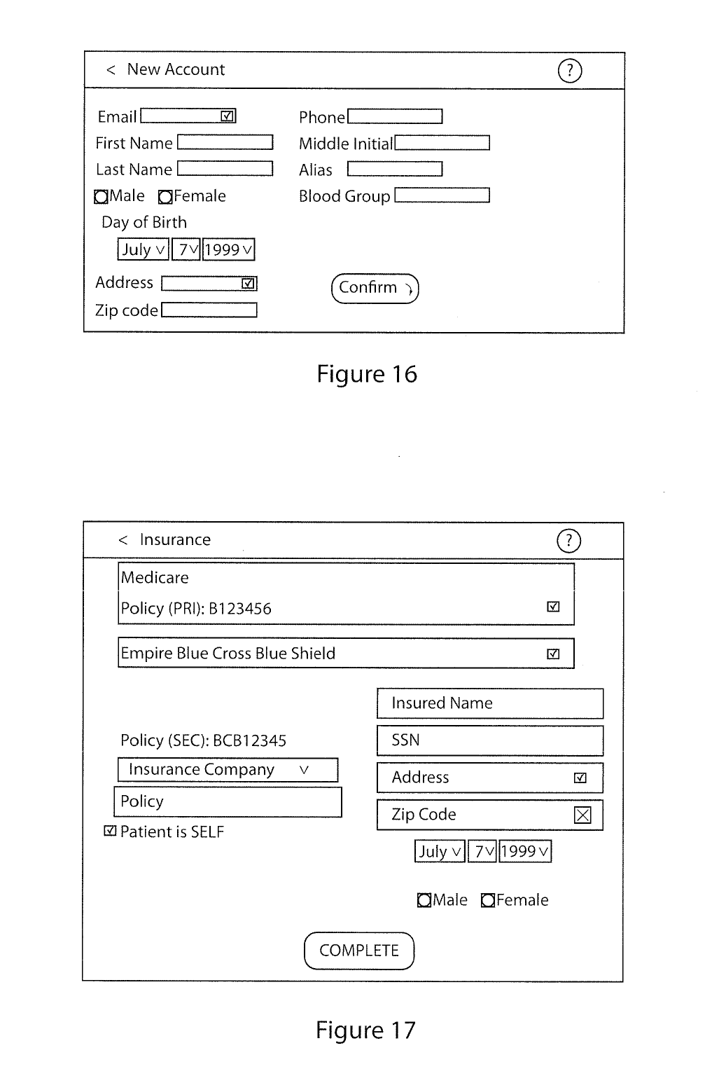

The entry of the patient data at the patient kioske portal of the modular medical clinic 100 may begin with a patient sign in. FIG. 15 is a screen shot of the initial screen displayed to the patient by the patient data collection system that is displayed on the touch screen computer 13, in which the patient may sign into the modular medical clinic 100 as a returning patient, or my register as a new patient. FIG. 16 is a screen shot of for registration of a new patient. In some embodiments, instead of a return patient registering using the sign in pages displayed on the touch screen computer 13, a patient may register using fingerprint or retinal scan identification. The collection of the registration data may continue with the entry of insurance information, as depicted in the screen shot illustrated in FIG. 17.

In some embodiments, the network may provide for past medical history of diagnosis and treatments of the patient by the network of modular medical clinics 100 to be included in the electronic medical records system, and may also autopopulate the records for the patient with data on past medical history from existing insurance claims based data, and health record companies, such as Microsoft Health Vault, MMR, prescription's databases (similar to ones used by Rite Aid), CCR, HIE and any other reliable sources.

The chief complaint of the patient may then be established. Non-limiting examples of such medical services, diagnoses, health advise, wellness advise and/or medical conditions that can be identified, treated and/or addressed in connection with the identification of the chief complaint include, but are not limited to: Acid Reflux; Hypertension Management; Allergies; Athlete's Foot; Acne; Mental Health Counseling; Wellness Counseling; Asthma; Cold Sores; Vaccinations; Arthritis; Bronchitis; Impetigo; Wellness Coaching; Weight Loss; Eating Disorders; Bladder Infections; Insect Stings; Allergic Reactions; Rashes; Hemorrhoids; Minor Burns; Health Risk Management; Migraine Headaches; Common Colds; Virus Infections; Bacterial Infections; Minor Skin Infections; Chronic Disease Management; Coughs; Poison Oak/Ivy; Diarrhea; Rashes; Diabetes; Ringworm; Lice; Ear Infections; Sties; Flu; Fever; Gout; Headache (minor); Pink Eye; Sinus Infections; Sore Throat; Ear Infections; Cramps; STDs; Strep Throat; Throat Infections; Feeding Problems For Newborns; Vomiting; Teething; Gastrointestinal Problems; Anxiety; Depression, Formula Advice For Newborns; Concussion; Head Injuries; Bone Fractures; Sprains; Hair Loss; Alopecia; Eye Infections; Urinary Tract Infections; Constipation; Appendicitis; Pharyngitis; Medication Therapy Management; Acid Reflux Disease; Acne; Alcohol abuse; Allergies; Antisocial Personality Disorder; Attention Deficit Disorder; Altitude Sickness; Alzheimer's Disease; Andropause; Anger management; Anorexia Nervosa; Arthritis; Aspergers Syndrome; Asthma; Autism; Back Pain; Bad Breath (Halitosis); Baldness; Bedwetting; Bipolar Disorder; Bladder Cancer; Body Dysmorphic Disorder; Bone Cancer; Brain Cancer; Breast Cancer; Brain Tumors; Brain Injury; Bronchitis; Burns; Bursitis; Cancer; Canker Sores; Carpal Tunnel Syndrome; Celiac Disease; Cervical Cancer; Cholesterol; Chronic Obstructive Pulmonary Disease; Colon Cancer; Congestive Heart Failure; Cradle Cap; Crohn's Disease; Dandruff; Deep Vein Thrombosis; Dehydration; Depression; Diabetes; Diaper Rash; Diarrhea; Disabilities; Diverticulitis; Down Syndrome; Drug Abuse; Smoking Cessation; Dysfunctional Uterine Bleeding; Dyslexia; Ear Infections; Ear Problems; Eating Disorders; Eczema; Endometriosis; Enlarged Prostate; Epilepsy; Erectile Dysfunction; Eye Problems; Fibromyalgia; Fracture; Gallbladder Disease; Gallstones; Generalized Anxiety Disorder; Genital Herpes; Genital Warts; Glomerulonephritis; Gonorrhea; Gout; Gum Diseases; Gynecomastia; Head Lice; Headache; Hearing Loss; Heart Attacks; Heart Disease; Heartburn; Heat Stroke; Heel Pain; Hemorrhage; Hemorrhoids; Hepatitis; Herniated Discs; Hiatal Hernia; HIV/AIDS; Hives; Hyperglycemia; Hyperkalemia; Hypertension; Hyperthyroidism; Hypothyroidism; Infectious Diseases; Infectious Mononucleosis; Influenza; Infertility; Insulin Dependent Diabetes Mellitus; Iron Deficiency Anemia; Irritable Bowel Syndrome; Irritable Male Syndrome; Itching; Joint Pain; Juvenile Diabetes1; Juvenile Rheumatoid Arthritis; Kidney Diseases; Kidney Stones; Leukemia; Liver Cancer; Lung Cancer; Mad Cow Disease; Malaria; Medication Management; Melena; Memory Loss; Menopause; Mesothelioma; Migraine; Miscarriages; Mucus In Stool; Multiple Personality Disorder; Multiple Sclerosis; Muscle Cramps; Muscle Fatigue; Muscle Pain; Nail Biting; Narcissistic Personality Disorder; Neck Pain; Obesity; Obsessive Compulsive Disorder; Osteoarthritis; Osteomyelitis; Osteoporosis; Ovarian Cancer; Ovarian Cyst; Pain; Pain Management; Panic Attack; Parkinson's Disease; Peripheral Artery Disease; Personality Disorders; Pervasive Developmental Disorder; Peyronie's Disease; Phobias; Pink Eye; Polio; Pneumonia; Post Nasal Drip; Post Traumatic Stress Disorder; Premature Baby; Premenstrual Syndrome; Prostate Cancer; Psoriasis; Reactive Attachment Disorder; Renal Failure; Restless Legs Syndrome; Rheumatoid Arthritis; Rheumatic Fever; Ringworm; Rosacea; Rotator Cuff; Scabies; Scars; Sciatica; Schizophrenia; Sexually Transmitted Disease; Sinus Infections; Skin Cancer; Skin Rash; Sleep Apnea; Sleep Disorders; Smallpox; Snoring; Social Anxiety; Staph Infection; Stomach Cancer; Strep Throat; Sudden Infant Death Syndrome; Sunburn; Syphilis; Systemic Lupus Erythematosus; Tennis Elbow; Termination of Pregnancy; Testicular Cancer; Tooth Decay; Tuberculosis; Ulcers; Urinary Tract Infection; Varicose Veins; Vertigo; Warts; Williams Syndrome; Yeast Infection; Yellow Fever; and combinations thereof.

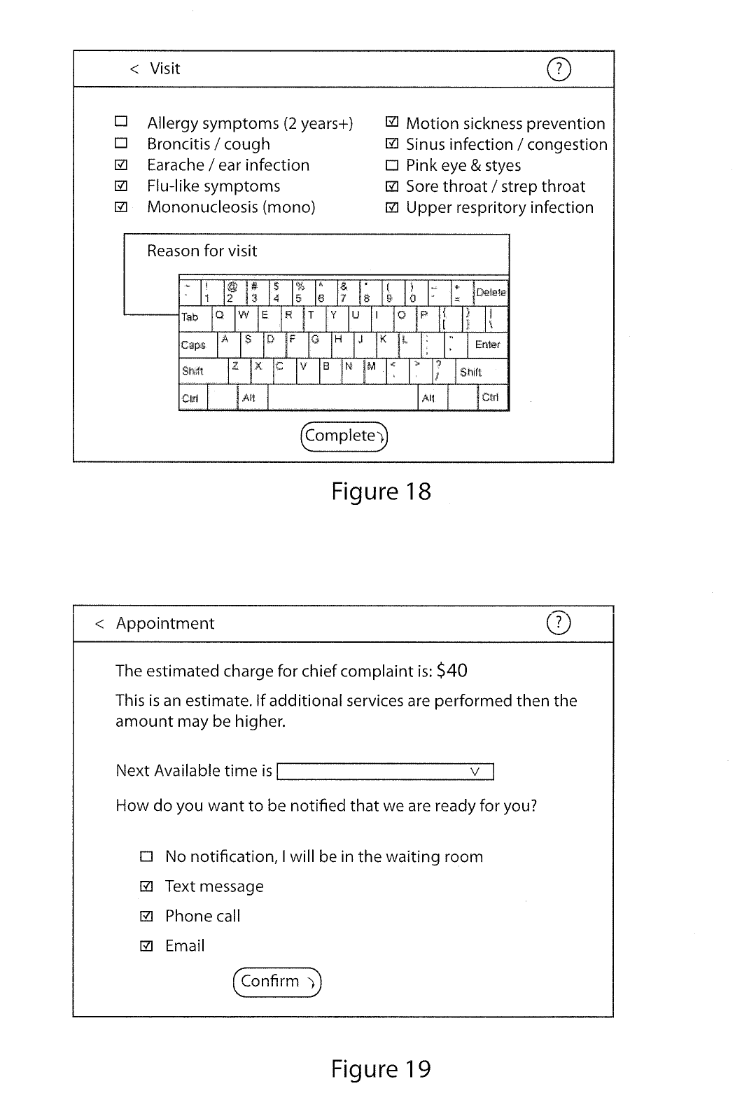

FIG. 18 depicts one embodiment of a screen shot from the touch screen computer 13, on which the patient may enter the chief complaint for which the visit to the modular medical clinic 100 is being made. Other preferences may also be entered by the patient including a preferred pharmacy.

FIG. 19 depicts the screen shot that displays the estimated charges for the treatment/diagnosis in response to the chief complaint to be charged by the patient for the visiting the modular medical clinic 100, as well as an estimated time for treatment/diagnosis of the patient.

The patient is also given an opportunity to select the means by which they are notified that the modular medical clinic 100 is available for their treatment/diagnosis.

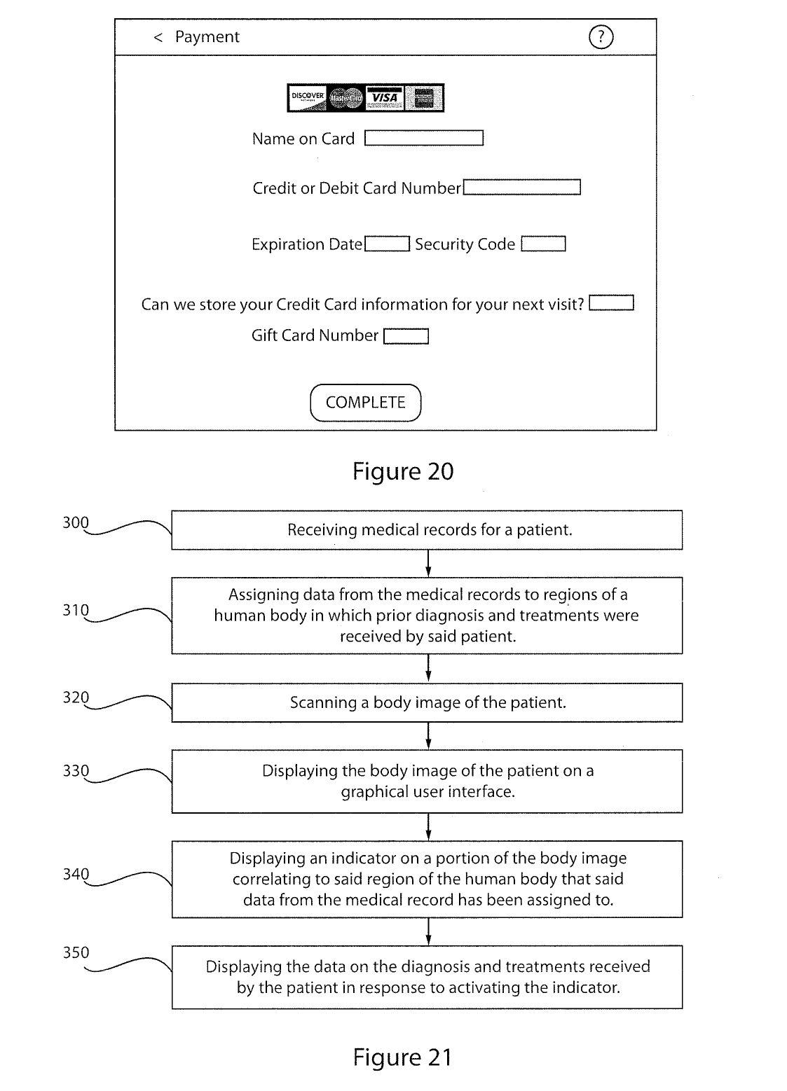

FIG. 20 depicts the screen shot for entry of the patient's payment method. The point of sale (POS) may also begin in the patient check in module 10, which allow for the collection of funds, such as payments in the form of credit, debit, check, insurance, and co-pays. The POS process allows for the distribution of such funds to the appropriate parties. This system should allow for parameters to be set up, bank routing information to be entered and for reporting of the dispersed funds to the appropriate parties. In insurance claim cases the system has the capability to file the claim under the doctors ID number on behalf. Credit card information may be collected at the patient check in module 10 using magnetic swipe. The patients credit card information may be saved as a data within the electronic medical record system. The entry of the patient data at the patient kioske portal of the modular medical clinic 100 may end following payment by the patient.

At any time that the patient is entering the patient data into the interface of the patient check in module 10, the patient may request instruction from a live remote agent, i.e., medical information collection personnel 120, via the telephone 14, e.g., video telephone. In some embodiments, a help button may be available on each of the patient data entry screens described above, in which by selecting the help button, the patient activates a portal to the remote live agent. In other embodiments, removing the handset from the receiver of the telephone 14 activates the portal to the remote live agent, e.g., medical information collection personal 120.

In some embodiments, the when the modular medical clinic 100 is ready to diagnosis the patient, the patient may receive a notice on their cellular phone, e.g., via text or phone call, or my receive a notice sent to their email. An identifier, such as the patient's name and/or an assigned patient number, may also be displayed on the display panels 12, i.e., display screens, that are present on the exterior of the modular medical clinic 100 when the modular medical clinic is available to receive the patient for diagnosis and/or treatment.

Referring to FIG. 14, at step 210 the patient enters the modular medical clinic 100, and may be escorted by an on site registered nurse, licensed practical nurse (LPN), or medical assistant. Based upon the chief complaint, if patient needs to put on a gown, a changing area is provided by the combination of the dressing module 35 and the curtain that can extend along tracks 4a, 4b. In some instances, the nurse may leave the modular medical clinic 100 while the patient is changing, wherein during these instances the security shutters 19, 38, 53 and 54 may secure the contents of the laboratory module 19, the inventory modules 35a, 35b, 35c and primary care module 15. Green and red lights on the exterior of the modular medical clinic 100 indicate to the nurse when the patient is changing and when they have finished.

At step 220 at the process flow depicted in FIG. 14, a scan is completed of the patient. For example, in some embodiments, the patient may be given instructions on the display 31 of the display module 30, which may be provided by an avatar depicted on the display, such as to stand on a designated floor tile, where the patient is scanned to capture a body image of the patient. In some embodiments, the designated floor tile is also a scale for taking the weight of the patient. In some embodiments, the patient may be scanned using wave millimeter technology/wave millimeter scanners.

As used herein, “wave millimeter technology” and “wave millimeter scanner” refers to a device for producing an image of patient's body, in which beams of radio frequency (RF) energy in the millimeter wave spectrum are projected over the body's surface from two antennas as they rotate around the body, wherein the RF energy is reflected back from the body and/or other objects contacted with the beams of radio frequency (RF) energy is used to construct a three-dimensional image. In some embodiments, the millimeter wave spectrum may range from 0.001 meters (1 millimeter) and 0.01 meters (10 millimeters).

Millimeter wave scanners typically produce their waves with a series of small, disc-like transmitters stacked on one another like vertebrae in a spine. A single machine may contain two of these stacks, each surrounded by a curved protective shell known as a randome, connected by a bar that pivots around a central point. Each transmitter emits a pulse of energy, which travels as a wave to a person standing in the machine, passes through the person's clothes, reflects off the person's skin or concealed solid and liquid objects and then travels back, where the transmitter, now acting like a receiver, detects the signal. Because there are several transmitter/receiver discs stacked vertically and because these stacks rotate around the person, the device can form a complete picture, from head to toe and front to back. Millimeter wave scanners themselves come in two varieties: active and passive. Active scanners direct millimeter wave energy at the subject and then interpret the reflected energy. Passive systems create images using only ambient radiation and radiation emitted from the human body or objects.

In some embodiments, the millimeter wave scanner 600 may be supported by an interior wall of the modular medical clinic 100, or may be supported by a ceiling of the modular medical clinic 100. In yet another embodiment, the millimeter wave scanner may extend from a compartment in the floor of the modular medical clinic 100. Typically, the millimeter wave scanner 600 is configured so that it can be stored in a position that does not obstruct diagnosis or treatment of the patient by the onsite nurse and/or the remote provider 130 after the body scan is completed.

Although the body scan has been described above using a millimeter wave scanner 600, other devices may be employed to create the three dimensional body scan of the patient. For example, the scan of the patient at step 220 of the method described in FIG. 14 may also be provided using a Microsoft Kinect system and/or backscatter machine.

Following the body scan of the patient, the on-site nurse may take the patients vitals including, for example, blood pressure, temperature, pulse oximetry (SpO2) and a combination thereof at step 230 of the method depicted in FIG. 14. The equipment used by the on-site nurse may be connected to the modular medical clinic 100, e.g., connected to the primary care panel 16 of the nurse station/primary care module 15. Once the vitals measurements of the patient has concluded the data may be sent to the network to be viewed by the provider 130. For example, once the vital measurements are completed, the on site nurse may hit a button indicating the vitals capture segment of the visit has been completed, and the collected vitals, body scan and other electronic medical records collected are sent over the network collection system 160 to the provider 130, at step 240 of the method depicted in FIG. 14. In one example, the Avatar that is depicted on the display 32 can announce to the patient “I will now connect you with the doctor”. The registered nurse can also enter the patient's vital information into the nurse station tablet computer 17 of the primary care module 15. The vitals information in addition to the information provided by the patient at the client check in may than be sent to the remote provider and remote provider systems 130.

The remote provider 130 can have access to the patients records in real time through the Doctor's portal. For example, the Doctor's portal may be accessed via a web site. The portal should help the provider 130 sign up on the front end and behind password protection should act as a dashboard for remote patient visits. Typically, this allows for medical records, video conferencing, diagnostic device feed, and account management, such as how many patients were seen, what reimbursements are pending and have paid, etc.

In some embodiments, forwarding the data from the electronic medical records system corresponding to the patient being treated by the modular medical clinic 100 to the provider 130 at step 240 of the process flow depicted in FIG. 14 may include assigning the data of the prior treatments and diagnosis of the patient that have been collected in the electronic medical records to regions of a human body in which prior diagnosis and treatments were received by the patient, in accordance with the method depicted in FIG. 21. FIG. 21 depicts a flow chart describing one embodiment of a method for displaying medical records. In some embodiments, the step of receiving medical records from a patient at step 300 may include the data in the electronic medical records system of the network including the modular medical clinic 100, which may include patient data entered at the check in modules 10 of the modular medical clinic. As indicated above, the electronic medical records system includes medical records for the patient recorded from prior visits to modular medical clinics 100 within the network, as well as medical records for the patient that can be received from other sources, such as the databases of insurance companies keeping electronic medical records of the patient.

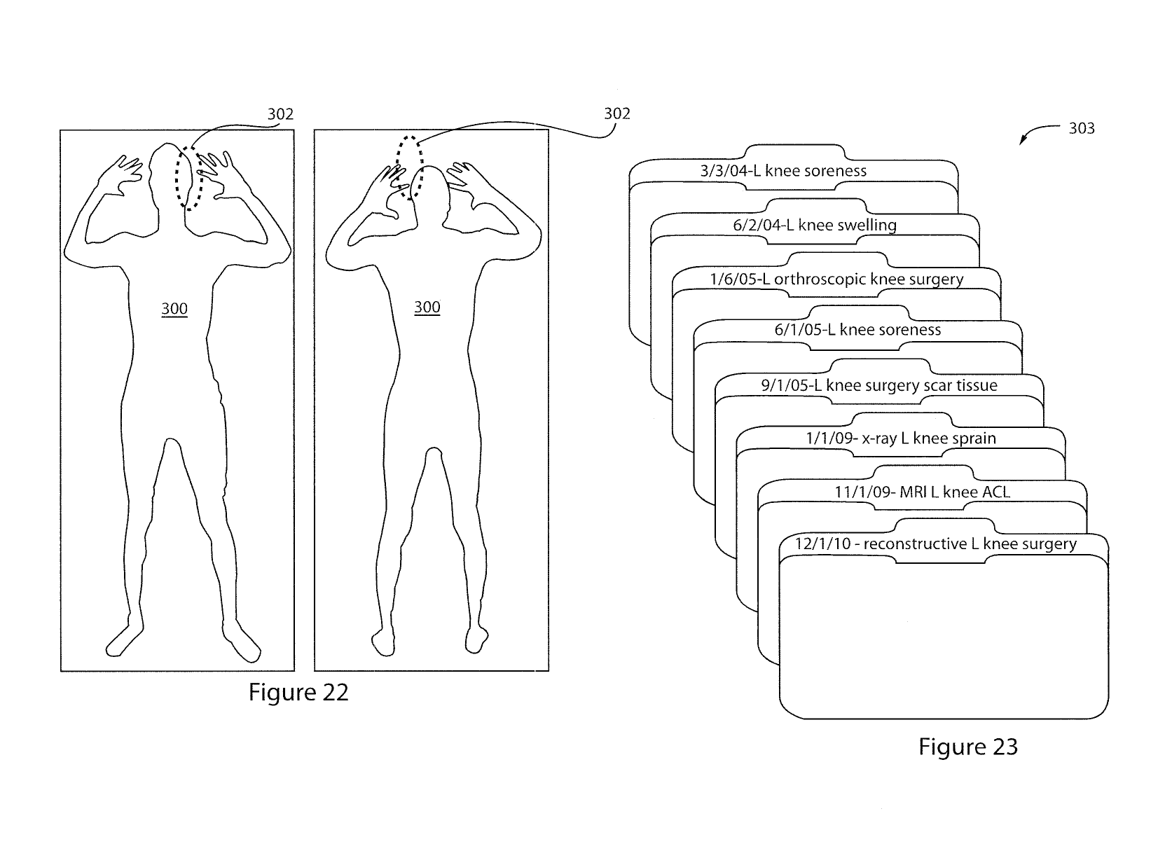

At step 310 of the method depicted in FIG. 21, data from the electronic medical records may then be assigned to regions of the body in which the prior diagnosis and treatments had been received by the patient. Step 320 of the of the method for displaying medical records, a body image of the patient may be scanned. Scanning the body image may be provided by wave millimeter technology, as described above. At step 330 of the method of displaying medical records that is depicted in FIG. 21, the scanned body image 300 may be depicted on a graphical user interface, as depicted in FIG. 22. At step 340 of the method depicted in FIG. 21, an indicator 301, 302 is displayed on the portions of the body image 300 correlating to the region of the human body that the data from the medical records for the patient has been assigned to, as depicted in FIG. 22.

FIG. 22 depicts one embodiment of a screen shot of a graphical user interface depicted on the computer screen of the provider's portal, i.e., provider's pod, to the modular medical clinic 100. FIG. 22 depicts one embodiment of the scanned body image 300 of the patient depicted on the screen of at least one of the first station 131 and the second station 132 of the provider's pod, which is used by the remote provider 130 to interact with the modular medical pod 100. In some embodiments, the indicator 301, 302, e.g., icon, may be positioned on each portion of the scanned body image 300 correlating to the region of the human body of the patient that the data from the electronic medical records has been assigned to.

At step 350 of the method depicted in FIG. 21, selecting the indicator 301, 302 on the graphical user interface displays the data on the diagnosis and treatments received by the patient for that region of the human body. For example, selecting the indicator 301, 302 on the graphical user interface depicted in FIG. 22 may open a new screen, i.e., window, including folders 303 including the data on the diagnosis and treatments received by the patient for the region of the human body, as depicted in FIG. 23. In some embodiments, the indicator 301, 302 may be color coordinated to indicate the number of diagnosis and/or treatments that a patient has received for that portion of the body. For example, in the embodiment that is depicted in FIG. 22, two indicators 301, 302 are depicted on the body scan of the patient. The first indicator 301 is positioned on the knee portion of the body scan of the patient, and the second indicator 302 is positioned on the ear portion of the body scan of the patient, indicating that the patient has received treatments and/or diagnosis related to both the knee and ear. In one example, if the patient has had a higher number of treatments and/or diagnosis for his or her knee than her ear, the first indicator 301 may be color coordinated to indicate to the provider viewing the screen of the body scan that the patient has had a number of treatments for his knee, e.g., may have a chronic knee pain etc. For example, to indicating the greater number of prior treatments received by the patient for his knee, the indicator 301 positioned over the knee portion of the body scan 300 may be red, whereas the indicator 302 positioned over the ear portion of the body scan 300 may be yellow. It is noted that this is only one color scheme that may be employed to indicate the frequency of treatments to a portion of the patients body, which is intended to be illustrative and not limiting. Any number of colors may be employed for any number of indicators that are positioned on the body scan to indicate prior medical history is within the scope of the present disclosure.

In some embodiments, the indicators 301, 302 may also be accompanied by text data, such as a number indicating number of treatments/diagnosis and/or the date of a most recent treatment/diagnosis, may also be displayed with the indicator 301, 302. In other embodiments, the indicator 301, 302 may also illustrate if a region of the body scan that prior medical history is available for is tied to the chief complaint that the patient has entered at the check in module 10 of the modular medical clinic 100.

FIG. 23 illustrates one embodiment of a screen shot depicted to the provider 130 after the provider 130 activates the first indicator 301. In this example, activating the first indicator 301 displays folders 303 including the data on the diagnosis and treatments received by the patient for the region of the human body. Each folder 303 may have a title summarizing the treatment and/or diagnosis that the patient received on a specific date. In some embodiments, each folder may have a title summarizing the treatment or diagnosis. The folders may also be organized by date. The folder's 303 may each include data relating to patient complaint, patient measurements, such as vitals, provider notes, medications prescribed, x-rays, and may include images directed to the treatment and/or diagnosis of the patient.

By clicking on the folders 303, i.e., activating the folders, the provider 130 may view the folder contents. The interface depicted in FIGS. 22 and 23 provides a means by which a provider 130 can access the contents of electronic medical records that are specific to a region of the body of the patient in real time. For example, instead of receiving a file on a patient that includes all of the diagnosis and treatments that the patient has received during their entire medical history, the methods, systems and apparatuses can provide only information specific to a body region that the provider would like to view without viewing the entire medical history of the patient. Therefore, providing the provider with an efficient means to view the information needed during a medical consultation.

At step 250 of the method depicted in FIG. 14, video conferencing may begin between the provider 130 and the modular medical clinic 100 through the network connection system 160. One embodiment of a screen shot of the provider portal to the examination room provided within the modular medical clinic is depicted in FIG. 24. More specifically, the provider 130 may view the patient within the modular medical clinic 100 through the video camera 32 of the display module 30 of the modular medical clinic 100. The provider 130 can control the pan and tilt angles of the video camera 32.

In some embodiments, the provider 130 will appear on the screen of the display 31 of the display module 30. The provider can also mock up the images on the display 31 of the misplay module 30 remotely; so that the patient within the modular medical clinic will be able to see the medical equipment info and video feed. At this point, the provider 130 may discuss the chief complaint with the patient, and run additional diagnostic tests administered through the on site nurse if necessary. The additional diagnostic tests may include cholesterol, glucose, stethoscope, otoscope and combinations thereof.

In some embodiments, the provider 130 may use a diagnostic system that provides clinical decision support in diagnosis the patient within the modular medical clinic 100. In one embodiment, the diagnostic system is employed in a method for diagnosing a patient that may employ artificial intelligence in combination with a database, which may be stored on the at least one server 110, in which the database couples prior provider diagnosis with medical device data including imagery collected via video stream and still images from exam cameras, and other devices such as otoscopes and stethoscopes, on all medical conditions that have been collected from patients through the modular medical clinic 100. Once a sufficient sampling of a condition resides in the database, artificial intelligence is applied to recognize medical device output against the database or control group. Classification ability will be further broken down by doctor, and healthcare entity. In some embodiments, the modular medical clinic 100 may be able to auto diagnose and/or assist in the diagnosis or medical conditions and diseases etc. based on the acquired knowledge and previously supplied medical input.

FIG. 25 is a block/flow diagram illustrating one embodiment of a method for providing medical diagnostics that employs artificial intelligence and a database of comparative data to provide an initial diagnostic for a measurement of a patient. The method may begin with providing the database comprised of comparative data collected from the plurality of patients at step 400. The comparative data is typically provided by the patients that are being diagnosed by the network including a plurality of modular medical clinics 100. The source of the comparative data may also be provided by other sources of medical history for the patients, which can include databases of health insurance claims, research databases and other databases of medical history. In some embodiments, the data may include images and videos of the physical symptom taken from a patient corresponding to an ailment, condition, disease, disorder, illness, malady, syndrome, poisonings, adverse effects of drugs & chemicals or combination thereof. Images and videos are not the only source of data that is suitable for the comparative data. For example, the source of comparative data may be any measurement that is taken from a patient in the administration of a diagnosis and/or treatment. For example, the comparative data database may include individual modules including, but not limited too, primary care, optical, cardiac and mental health.

In some embodiments, each value of comparative data includes a diagnosis code designating a diagnosis for an ailment, condition, disease, disorder, illness, malady, syndrome, poisonings, adverse effects of drugs & chemicals (hereafter collectively referred to as ailment) corresponding to the data taken during the diagnosis/treatment of the patient. The diagnostic code is human assigned. The diagnostic codes may be selected to correspond to an ailment, condition, disease, disorder, illness, malady, syndrome, poisonings, adverse effects of drugs & chemicals, injuries and other reasons that a patient may seek treatment at the modular medical clinic. Diagnostic coding is the translation of written descriptions of diseases, illnesses and injuries into codes. Although not necessary, and sometimes not preferred, in some embodiments, the diagnostic coding may include coding criteria from medical classification systems, such as ICD-9-CM (volumes 1 and 2), ICD-10, ICPC-2, International Classification of Sleep Disorders, NANDA, Diagnostic and Statistical Manual of Mental Disorders, Mendelian Inheritance in Man, Read code, SNOMED and a combination thereof. It is noted that the above list is provided for illustrative purposes only and is not intended to be an exhaustive list. In some embodiments, each value of comparative data is tagged with an institution type designation. The institutional type designation indicates the identity of the entity that assigned a diagnosis designated by the diagnostic coding for each value of comparative data. The institutional type designation may be human assigned.

Referring to FIG. 25, a measurement may be taken from a patient at step 410. The measurement of the patient is typically taken while the patient is present in the modular medical clinic 100 using at least one of the medical devices that are in communication with the network including the modular medical clinic 100. For example, the measurement may be an image of physical symptom of the patient taken using an endscope, dermoscope, x-ray, wave millimeter scan, otoscope, exam camera, exam video, ENT scope and combinations thereof. It is noted that image data is not the only type of data may be employed in this method. As long as the data of the measurement taken from the patient has a corresponding type of comparative data is present in the comparative data database, the diagnostic method may apply. For example, the measurements taken from the patient suitable for use with the method described in FIG. 4 may include measurements from a digital stethoscope, spirometer, ECG/EKG and others. Measurement data may then be extracted from the measurement of the patient for use for comparison with the data that is included in the comparative data database at step 420.

In a following step, the measurement data may be compared to the comparative data in the database by employing artificial intelligence at step 460 of the method depicted in FIG. 25. A match determined by the artificial intelligence between the measurement data and the comparative data designates the measurement data with the diagnostic code as an initial diagnosis. In one embodiment, the artificial Intelligence (AI) is the ability of computer software and hardware to searching, i.e., finding “good” data after having been provided only limited direction, especially from a large quantity of available data, and/or to recognize patterns, i.e., finding items with similar characteristics, or identifying an entity when not all its characteristics are stated or available. For example, the artificial intelligence engine that is employed at step 460 of the method depicted in FIG. 35 may take the measurement data from the patient and search the comparative data in the comparative data database to find potential matches. In some embodiments, to find potential matches between the measurement data and the comparative data the artificial intelligence engine may employ pattern recognition abilities. In some embodiments, the artificial intelligence engine may include neural networks, expert systems, genetic algorithms, intelligent agents, logic programming, and fuzzy logic. Neural network artificial intelligence is based loosely upon the cellular structure of the human brain. Cells, or storage locations, and connections between the locations are established in the computer. As in the human brain, connections among the cells are strengthened or weakened based upon their ability to yield “productive” results. The system uses an algorithm to “learn” from experience. Neural nets are an inductive reasoning method. Expert systems are usually built using large sets of “rules.” Genetic algorithms utilize fitness functions, which are relationships among criteria, to grade candidates. For example, a genetic algorithm may: 1. evaluate the population against “high fitness” criteria; 2. if a candidate in the population meets the criteria stop, else; 3. select the best of the current set using a selection strategy and diversity maintenance, then; 4. reproduce using crossover & mutation, and 5. return to 1. It is noted that the above description of artificial intelligence is provided for illustrative purposes only and is not intended to limit the present disclosure.

The initial diagnosis made using the artificial intelligence engine may indicate the diagnostic code for the ailment, and also include an expression of the number of matches for the comparative data to measurement data by said institution type designation. For example, if the measurement data taken from the patient matches 100 cases in the comparative data database, a box indicating that 100 comparative cases may be displayed to the provider 130. The initial diagnosis including the expression of the number of matches for the comparative data to measurement data by the institution type designation may then be displayed in step 460 of the method depicted in FIG. 25. The institution type designation may indicate whether comparative diagnosis was provide by specialists in the area of the ailment being diagnosed and treated. The institution type designation may also designate highly rated groups of providers and hospitals where applicable. Further, the institution type designation may also designate a specific provider that has made the diagnosis in the comparative data. For example, if a specific provider is known for treating a specific illness, that information may be displayed as part of the institution type designation depicted as part of the initial diagnosis.

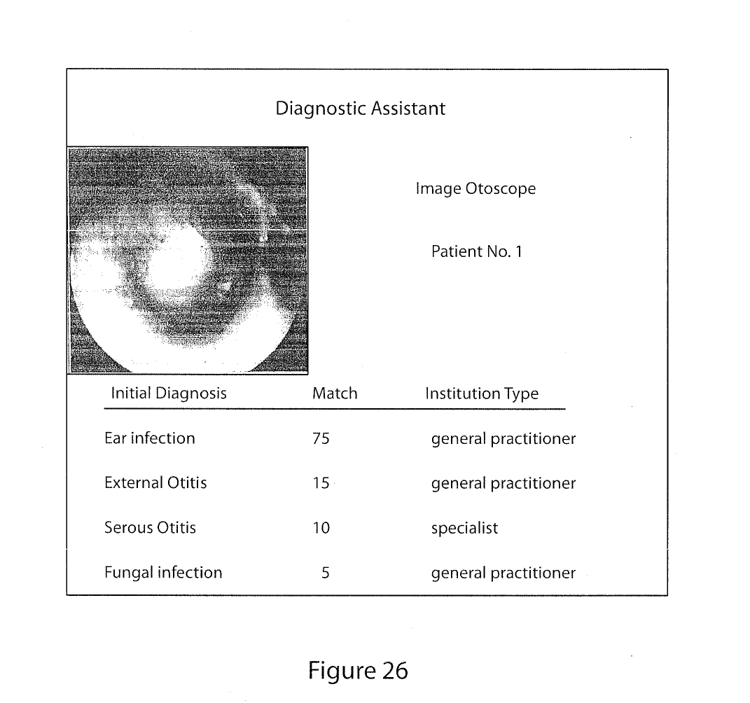

FIG. 26 depicts one embodiment of a screen shot of a preliminary diagnostic displayed on one of the displays to the providers pod, i.e., portal to the modular medical clinic 100, that is produced in response to an image of a sore ear taken from a patient. The image is taken using a digital otoscope that is in communication with the primary care panel 16 of the nurse station/primary care module 15. In the example depicted in FIG. 26, the image taken from the digital otoscope for the patient being examined in the modular medical clinic 100 matched 75 comparative data entries within the comparative data database, in which the comparative data entries indicated that a similar image had been diagnosed as an ear infection. The image taken from the digital otoscope for the patient being examined matched 15 comparative data entries within the comparative data database, in which the comparative data entries indicated that a similar image had been diagnosed as an external otitis. The image taken from the digital otoscope for the patient being examined matched 10 comparative data entries within the comparative data database, in which the comparative data entries indicated that a similar image had been diagnosed as an serous otitis. The image taken from the digital otoscope for the patient being examined matched 5 comparative data entries within the comparative data database, in which the comparative data entries indicated that a similar image had been diagnosed as a fungal infection. Although the diagnosis of an ear infection is associated with a greater number of diagnosis, it is noted that in this example an ear specialist is included in the types of institutions that provided diagnosis for data comparative to the data, e.g., image of ear, taken by the patient. In this example, the best practices may be the diagnosis by the ear specialist in comparison to the other diagnosis's provided by the general practitioners.

It is noted that the example described above with reference to FIG. 26 is only one example of a diagnosis that may be provided using the modular medical clinic 100, and that the methods, apparatuses and systems disclosed herein are not limited to only this example. For example, methods have been contemplated in which changes in a patient's body is correlated to comparative data using artificial intelligence.

In one embodiment, a diagnostic system that provides clinical decision support in diagnosis the patient within the modular medical clinic 100 may also be provided in which millimeter wave scan technology is used to measure changes in the body of a patient over time. In this embodiment, a database comprised of comparative data is provided wherein each data value in the database correlates a measured change in body dimensions and symptoms of a patient to a diagnostic code. Each value of comparative data is tagged with an institution type designation. This database is similar to the database that has been described above with reference to step 400 of the method depicted in FIG. 25. In a following step, at least one symptom from the patient seeking treatment is recorded with the network. For example, the patient may have symptoms of an unusual lump or swelling on their body; a change in the size, shape or color of a mole; a sore that is slow to heal; a mouth or tongue ulcer that lasts longer than three weeks; a cough or croaky voice that lasts longer than three weeks; persistent difficulty swallowing or indigestion; problems passing urine; blood in patient urine and bowel motions; unexplained weight loss or heavy night sweats; an unexplained pain or ache that lasts longer than four weeks; breathlessness; coughing up blood and a combination thereof.

Changes in a patient's body geometry may be measured using a wave millimeter scanner. In some embodiments, the wave millimeter scanner that is used for measuring changes in the patient's body geometry is similar to the wave millimeter scanner employed to provide the body scan that is described above with reference to FIGS. 21 and 22. Therefore, the description of the wave millimeter scanner that is described above with reference to FIGS. 21 and 22 is suitable for the description of the wave millimeter scanner that is used to measure changes in the patient's body geometry. In some examples, changes in the patient's body geometry may include at least one of lumps and swellings in your neck, armpit, abdomen, groin or chest area of the patient. The changes in the patient's body geometry may be measured over multiple visits to the modular medical clinic 100, in which the first body scan of the first visit at the modular medical clinic 100 may provide the baseline of the measurements for the following visits to the modular medical clinic 100.

In some embodiments, artificial intelligence may be employed to compare the changes in the patient's body geometry and at least one patient symptom to the comparative data in the database. A match determined by the artificial intelligence between the changes in a patient's body geometry and the at least one patient symptom and the comparative data designates an initial diagnosis. The application of the artificial intelligence is similar to the application of the artificial intelligence that is described above with reference to step 450 of the method described with reference to FIG. 26. The initial diagnosis includes an expression of the number of matches for the comparative data to measurement data by said institution type designation. In the above example, in which the changes to the patients' body geometry may include at least one of lumps and swellings in your neck, armpit, abdomen, groin or chest area of the patient, the initial diagnosis may be a form of cancer.

It is noted that the above descriptions of diagnostic assistance for the provider 130 are optional, and may be omitted from the methods of treating the patient using the modular medical clinic 100.

During the diagnosis/treatment of the patient within the modular medical clinic 100, an on site nurse may collect all necessary supplies by working with an inventory control system that is integrated with the network including the modular medical clinic 100. In some embodiments, the inventory control system may include a bin system, bulk inventory system, auto increment and decrement inventory. These structures may be integrated into an inventory supply module 35a, 35b, 35c.

The on site nurse may administer some treatments within the modular medical clinic 100 under the instruction of the remote provider 130. In some embodiments, the on site nurse will collect blood if necessary, run any local tests or prepare to send specimen to the outside lab. In some embodiments, the provider 130 may select a diagnostic code corresponding to a treatment or diagnosis procedure of the patient. The diagnostic code designated a pick list of inventory that is need for the on site nurse to administer the treatment or diagnosis of the patient. The pick list includes numbers that correspond to medical supplies, i.e., inventory, that the on site nurse will need to administer the treatment, in which each number corresponds to a type of medical supply. The pick list may be displayed to the on site nurse on the nurse tablet computer 17. The numbers of the pick list designate inventory that is present in the bins 36 of the supply modules 35a, 35b, 35c. Each bin 36 is designated with a number indicating it's contents consistent with the numbering used in the pick list. Further, in some embodiments, when the remote provider designates a diagnostic code, the bins 36 containing items on the pick list for the diagnostic code may be illuminated. The illumination of the bins 36 indicates to the on-site nurse what supplies she will need to administer the diagnosis and/or treatment of the patient while under the supervision of the remote provider 130. Selection of the diagnostic code by the provider 130 also indicates a change in inventory of the items listed in the pick list that is present within the modular medical clinic 100. That change in inventory information is forwarded to the supply personnel and systems 140 through the operations personnel and systems 150.

If a script is to be issued to the patient being treated in the modular medical clinic 100, the provider 130 may discuss the script with the patient over the video display of the displayer module 130, and then e-script the prescription to the predefined pharmacy or print out the receipt locally at the modular medical clinic if requested by the patient. The provider 130 may also send the appropriate patient education to the printer present at the modular medical clinic 100. If a follow up visit is necessary, the provider 130 may schedule the visit, while the patient is still within the clinic.

At step 260 of the method depicted in FIG. 14, the provider 130 ends video conferencing with the patient.

At step 270 of the method depicted in FIG. 14, the check process may begin. In some examples, the Avatar or live agent appears on screen of the displayer module and goes through the patient check out process. Based on the procedures done during the visit the actual charge or co-pay will display on the screen. Patient will be requested to sign off on the charge on the nurse station tablet computer. A receipt will print out on onsite print for the patient. Online patient portal access instruction including user name and password info may also be provided to the patient. The patient will then be escorted from the modular medical clinic 100, so that it may be prepared for the next patient. The patient file may then be sent for processing.

A follow up mechanism can be put in place to deal with lab results, follow up visits, follow up information. This information may be accessible by the patient over on the website portal to the network.

The revenue cycle management of the modular medical clinic 100 and network may begin, which can include setting up a lock box account. Lock box accounts provide a way for companies to coordinate their account activities within one central location. For example, the insurance reimbursement for the payment received from the patient from the treatment/diagnosis in the modular medical clinic 100 may be deposited into a lockbox account and then allocated to at least 3 entities automatically, e.g., the provider 130, the operations personnel 150, and the owner of the modular medical clinic 100.

The modular medical clinic and network can be built in areas that could not previously support a clinic due to the lack or medical infrastructure in the area. The modular medical clinic allows for the redistribution of medical resources away from metropolitan areas that have an oversupply to areas classified as provider shortage areas. In one application, the modular medical clinic and associated network may be applied to a workman's compensation type application. In this application, the modular medical clinic 100 may be constructed on a job site, or close to a jobsite. A baseline of a worker fitness and health may be taken prior to the workers' starting a job. If the worker is hurt on the job, the worker may be examined in the modular medical clinic to determine if the injury is a new injury, an injury suffered prior to starting work at the construction site, or an aggravation of a prior injury.

In some embodiments, the methods, systems and apparatuses disclosed herein can significantly lower the cost for providing medical care. In comparison to conventional care clinics, the modular medical clinics and network disclosed herein can reduce costs by approximately half.

Any combination of one or more computer readable medium(s) may be utilized in combination with the methods and process flows disclosed herein. In some embodiments, the computer readable medium is non-transitory. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any programming language or any combination of one or more programming languages, including an object oriented programming language such as Java, Smalltalk, C++ or the like or a procedural programming language, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus or other devices provide processes for implementing the functions/acts specified in the flowchart and/or block diagram block or blocks.

The terms “computer system” and “computer network” as may be used in the present application may include a variety of combinations of fixed and/or portable computer hardware, software, peripherals, and storage devices. The computer system may include a plurality of individual components that are networked or otherwise linked to perform collaboratively, or may include one or more stand-alone components. The hardware and software components of the computer system of the present application may include and may be included within fixed and portable devices such as desktop, laptop, server. A module may be a component of a device, software, program, or system that implements some “functionality”, which can be embodied as software, hardware, processor or microprocessor, firmware, electronic circuitry, or etc,

While the present disclosure has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A modular medical clinic system for providing medical diagnostics comprising:
a patient check in module comprising an interface for receiving patient data from a patient, the patient check in module including video and audio systems for live communication with a remote information collection personnel;
a display module comprising a video display interface for depicting a remote provider in real time to the patient within the modular medical clinic and depicting the patient in real time to the remote provider; and
an inventory supply module with an inventory interface for receiving data on supplies used in the modular medical clinic.

2. The modular medical clinic system of claim 1 further comprising a primary care module comprising a medical device selected from the group consisting of otoscopes, stethoscopes, exam camera, thermometers, oximeter, blood pressure cuff, electrocardiogram, spirometer, transducer, Wood glucose measuring devices, scan devices and combinations thereof.

3. The modular medical clinic system of claim 1, wherein the inventory interface is though operations personnel and systems used to monitor the modular medical clinic system.

4. The modular medical clinic system of claim 3, wherein the inventory interface is in communication with a remote supplier for maintaining inventory.

5. A modular medical clinic system comprising:
a plurality of modules;
at least one sensor taking at least one measurement from at least one module of the plurality of modules; and
an interface with said at least one sensor for sending data from said at least one measurement to a remote operator.

6. The modular medical clinic system of claim 5, wherein the plurality of modules is selected from the group consisting of a patient check in module, a display module, an inventory supply module, primary care module, a laboratory module and a combination thereof.

7. The modular medical clinic system of claim 5, wherein the at least one measurement sent by the at least one sensor is a measurement of functionality of a system selected from the group consisting of a medical device connected to the primary care module, a laboratory device connected to the laboratory module, a display from the display module, a video from the video module, lighting of the modular medical clinic system, an electrical panel of the modular medical system, and combinations thereof.

8. The modular medical clinic system of claim 5, wherein the display module comprising a video display interface for depicting a remote provider in real time to a patient within the modular medical clinic and depicting the patient in real time to the remote provider, wherein video conferencing between the remote provider and the patient is administered by the remote operator.

9. The modular medical clinic system of claim 5, wherein the primary care module comprises a medical device connected to the interface provided by the remote operature, wherein the medical device is selected from the group consisting of otoscopes, stethoscopes, exam camera, thermometers, oximeter, blood pressure cuff, electrocardiogram, spirometer, transducer, blood glucose measuring devices, scan devices and combinations thereof.

10. A diagnostic system comprising:
a database comprised of comparative data collected from a plurality of patients, wherein each value of comparative data includes diagnosis for an aliment that is categorized by a diagnostic code, wherein said each value of comparative data is tagged with an institution type designation;
a diagnostic tool for taking a measurement of a patient;
a comparison tool for extracting measurement data from the measurement of the patient and comparing the measurement data to the comparative data in the database by employing artificial intelligence, wherein a match determined by the artificial intelligence between the measurement data and the comparative data designates the measurement data with the diagnostic code as an initial diagnosis, wherein the initial diagnosis includes an expression of the number of matches for the comparative data to measurement data by said institution type designation; and
a display depicting the initial diagnosis including the expression of the number of matches for the comparative data to measurement data by said institution type designation.

11. The method of claim 10, wherein the diagnostic tool is selected from the group consisting of a wave millimeter scanner, a backscatter machine, an x-ray, an otoscope, a stethoscope, an exam camera, a video, a thermometers, an oximeter, a blood pressure cuff, an electrocardiogram, a spirometer, a transducer, a blood glucose measuring devices, scan devices and a combination thereof.

12. The method of claim 10, wherein the artificial intelligence comprises neural networks, expert systems, genetic algorithms, intelligent agents, logic programming, fuzzy logic, or a combination thereof.

13. A method of diagnosis a patient:
providing a database comprised of comparative data, wherein each data value in the database correlates a measured change in body dimensions and symptoms of the patient to a diagnostic code;
recording at least one symptom from a patient;
measuring changes in a patient's body geometry using a wave millimeter scanner; and
comparing the changes in a patient body geometry and at least one patient symptom to the comparative data in the database by employing artificial intelligence, wherein a match determined by the artificial intelligence between the changes in a patient's body geometry and the at least one patient symptom and the comparative data designates the measurement data with the diagnostic code as an initial diagnosis.

14. The method of claim 13, wherein said each value of comparative data is tagged with an institution type designation and the initial diagnosis includes an expression of the number of matches for the comparative data to measurement data by said institution type designation.

15. The method of claim 13, wherein said measuring said changes in a patient's body geometry using said wave millimeter scanner is within a first modular medical clinic connected to a network of clinics, and wherein at least one of said each data value in the database comprised of said comparative data is obtained from a second modular medical clinic in said network of clinics.

16. A method of displaying medical records comprising:
receiving medical records for a patient;
assigning data from the medical records to regions of a human body in which prior diagnosis and treatments were received by said patient;
scanning a body image of the patient;
displaying the body image of the patient on a graphical user interface; and
displaying an indicator on a portion of the body image correlating to said region of the human body that said data from the medical record has been assigned to, wherein selecting said indicator on the graphical user interface displays said data on said diagnosis and treatments received by said patient for that region of the human body.

17. The method of claim 16, wherein the scanning the body image comprises a wave millimeter scanner.

18. The method of claim 16, wherein the indicator comprises a signal illustrating the frequency of treatments from said data to said region of the human body.

19. The method of claim 16, wherein the selecting of the indicator opens a new frame on the graphical user interface containing a plurality of folders containing said data.

20. A medical diagnostic system comprising:
at least one device taking at least one measurement from a patient; and
an interface including a first connection to a display for providing video conferencing, a second connection for receiving data from the at least one sensor, and a third connection to a remote operator, wherein data from the at least one sensor is forwarded to the remote operator, wherein the remote operator communicates information from that at least one sensor to a provider, and provides said video conferencing between the patient and a provider for medical diagnosis.

21. The medical diagnostic system of claim 20, wherein the at least one device is selected from the group consisting of an otoscope, a wave millimeter scanner, a backscatter machine, an x-ray, an otoscope, a stethoscope, an exam camera, a video, a thermometers, an oximeter, a blood pressure cuff, an electrocardiogram, a spirometer, a transducer, a blood glucose measuring devices, scan devices and a combination thereof.

22. The medical diagnostic system of claim 20, wherein the display comprises a game console, a mobile device, a cellular device, or a personal computer having a web cam.
